(19) United States
(12) Patent Application Publication
(10) Pub. No.: US 2025/0376522 A1
(43) Pub. Date: Dec. 11, 2025

(54) Targeting Cells with a Combination of CXCR2 Inhibition and CD47 Blockade

(71) Applicant: The Board of Trustees of the Leland Stanford Junior University, Stanford, CA (US)

(72) Inventors: Allison Banuelos, Stanford, CA (US); Allison Zhang, Stanford, CA (US); Kristopher D. Marjon, San Jose, CA (US); Hala Berouti, Stanford, CA (US); Irving L. Weissman, Sr., Stanford, CA (US)

(21) Appl. No.: 18/876,613

(22) PCT Filed: Jul. 6, 2023
(86) PCT No.: PCT/US2023/026983

§ 371 Date: Dec. 18, 2024

Related U.S. Application Data
(60) Provisional application No. 63/359,099, filed on Jul. 7, 2022.

(51) Int. Cl. C07K 16/28; A61K 39/00; A61K 45/06; A61P 35/00

(52) U.S. Cl. CPC C07K 16/2803; A61K 45/06; A61P 35/00; A61K 2039/505

(57) ABSTRACT

Methods are provided for targeting cells for depletion, including without limitation tumor cells such as solid tumor cells, in a regimen comprising contacting the tumor and immune effector cells with an effective dose of an anti-MSDC agent that reduces the abundance, immunosuppressive activity, or tumor recruitment of CXCR2⁺ granulocytic-myeloid derived suppressor cells, for example, an inhibitor of CXCR2; in combination with an effective dose of an inhibitor of CD47/SIRPα signaling.

### CROSS REFERENCE TO OTHER APPLICATIONS

This application claims the benefit of U.S. Provisional Application No. 63/359,099, filed Jul. 7, 2022, the contents of which are hereby incorporated by reference in its entirety.

### BACKGROUND

Over the last decade, advances in immunotherapy have revolutionized cancer treatment and have reignited the field of tumor immunology. Despite numerous clinical successes, many patients demonstrate varying responses to immunotherapies, and some types of cancers show almost complete resistance. While tremendous efforts have focused on T cell-mediated therapies, understanding the role of the innate immune system in regulating tumor progression has now come into sharper focus.

Macrophages, which are present in virtually all tissues, are responsible for executing homeostatic tasks, such as recognizing and clearing dying or unwanted cells through programmed cell removal, a process that is critical for maintaining tissue integrity. However, cells that express CD47, including tumor cells, can inhibit phagocytosis and escape immune surveillance.

Macrophages within the tumor microenvironment, termed tumor-associated macrophages (TAMs), are a major component of infiltrating leukocytes and can be found to exert immunosuppressive, or M2-like, phenotypes in the tumor. Other innate immune subsets found in tumors, such as myeloid-derived suppressor cells (MDSCs) have also been shown exhibit pro-tumorigenic activities. Frequencies of TAMs and MDSCs are closely associated with therapeutic resistance and poor prognosis. The discovery of TAMs and MDSCs and their immunosuppressive capabilities has fueled emerging efforts to explore modulation of myeloid cells for cancer immunotherapy.

Colony-stimulating factor-1 receptor (CSF1R) is a class III protein tyrosine kinase expressed on cells belonging to the mononuclear phagocyte lineage, including TAMs. Binding of CSF1 or IL-34 ligands activates signaling that is crucial for macrophage development, differentiation, and survival. The inhibition of TAM proliferation and survival through CSF1R blockade has been widely explored as a cancer immunotherapy. In pre-clinical models, CSF1R inhibition with monoclonal antibodies or small molecule antagonists demonstrate robust reduction of TAMs. Likewise, in the clinic, CSF1R inhibition also shows a reduction of macrophages in solid tumors. However, the reported effects of TAM depletion by CSF1R inhibitors show minimal anti-tumor efficacy and limited therapeutic benefits. Improving our understanding of the effects of CSF1R inhibition is necessary for developing and improving myeloid-target immunotherapies.

### SUMMARY

Methods are provided for targeting cells for depletion, including without limitation tumor cells such as solid tumor cells, in a regimen comprising contacting the tumor and immune effector cells with an effective dose of an anti-MSDC agent that reduces the abundance, immunosuppressive activity, or tumor recruitment of CXCR2⁺ granulocytic-myeloid derived suppressor cells, for example, an inhibitor of CXCR2; in combination with an effective dose of an inhibitor of CD47/SIRPα signaling. In some embodiments the contacting is performed in vivo. In some embodiments, the combination of agents provides a synergistic effect relative to the administration of the inhibitor of CXCR2, or the inhibitor of CD47/SIRPα signaling administered as a monotherapy. In various embodiments, the combination of agents is administered in a therapeutic regimen that may include conventional treatment, e.g. targeted anti-tumor antibodies, chemotherapy, radiation therapy, surgery, and the like. In other embodiments the combination of agents is used in the treatment of inflammatory disease associated with myeloid-derived suppressor cells, including without limitation peritonitis.

A benefit of the present invention can be the use of lowered doses of the agents relative to the dose required as a single agent. A benefit of the present invention can also, or alternatively, be a decrease in the length of time required for treatment, relative to the length of time required for treatment as a single agent. A benefit of the present invention can also, or alternatively, be an enhanced response relative to the response observed after treatment with a single agent.

In some embodiments, treatment of an individual with an inhibitor of CXCR2 in combination with an effective dose of an inhibitor of CD47/SIRPα signaling results in a reduction of the number of granulocytic-myeloid derived suppressor cells (G-MDSC) present in the tumor microenvironment of the treated individual, e.g. a reduction of at least about 10%, 20%, 30%, 40%, 50%, 60%, 70%, 80%, 90% 95% or more relative to the number of G-MDSC present in the absence of treatment. Mouse G-MDSCs may be characterized as CD11b+Ly6G Ly6C^{lo} cells. The counterpart human cells may be characterized as CD11b+CD15+CD14−CD33+/lo CD66b+cells.

An anti-CD47 agent for use in the methods of the disclosure interferes with binding between CD47 present on the cancer cell and SIRPα present on a phagocytic cell. Such methods increase phagocytosis of the cancer cell. Suitable anti-CD47 agents include soluble SIRPα polypeptides; soluble CD47; anti-CD47 antibodies, anti-SIRPα antibodies, and the like, where the term antibody encompasses antibody fragments and variants thereof, as known in the art. In some embodiments the anti-CD47 agent is an anti-CD47 antibody. In some embodiments the anti-CD47 antibody is a non-hemolytic antibody. In some embodiments the antibody comprises a human IgG4 Fc region.

Small molecule inhibitors of CXCR2 are known in the art; and may find use in the methods of the disclosure. In some embodiments the inhibitor is orally administered. In some embodiments the inhibitor is selected from, for example, Navaraxin, SB225002, SB265610, AZD5069, Danirixin, Reparixin, SX-682, Elubirixin, NSC 157449, MK-7123, and QBM076. In other embodiments a CXCR2 inhibitor is a large molecule, e.g. antibody or fragment thereof, that specifically binds to CXCR2.

### BRIEF DESCRIPTION OF THE FIGURES

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIGS. 1A-1C. A) Mating scheme by which inducible CSF1R knockout mice were generated and diagram illustrating the induction of Cre expression and subsequent recombination after Poly I:C administration. B) Schematic depicting timeline of Poly I:C injections to delete CSF1R. CSF1R^{FI/FI}-Mx1-Cre mice received 100 ug doses of Poly I:C every other day for a total of three doses. Poly I:C was administered through intraperitoneal injections. Tissues were harvested one week after injections were completed. C) Representative flow cytometry plots of CSF1R cell surface expression on CD11b+ cells within the bone marrow, peripheral blood, peritoneal cells, and spleen one week after Poly I:C administration in CSF1R^{FI/FI}-Mx1-Cre mice, compared with control mice (n=5 mice).

FIGS. 2A-2D. A) Schematic depicting experimental timeline. CSF1R^{FI/FI}-Mx1-Cre mice received three intraperitoneal injections of Poly I:C (100 ug) and were rested for one week. Control and Poly I:C-treated CSF1R^{FI/FI}-Mx1-Cre mice were then infused intraperitoneally with thioglycolate broth. Peritoneal cells were harvested 48 hours following thioglycolate broth infusion. B) Representative flow cytometry plots of CSF1R expression CD11b+ peritoneal cells and 48 hours after thioglycolate broth infusion in control and Poly I:C-treated CSF1R^{FI/FI}-Mx1-Cre mice (n=5 mice). C) Representative flow cytometry plots of F4/80+ macrophages 48 hours after thioglycolate broth infusion in control and Poly I:C-treated CSF1R^{FI/FI}-Mx1-Cre mice (n=5 mice). Representative flow cytometry plots of peritoneal cells 48 hours after thioglycolate broth infusion of total CD11b+ cells, M-MDSCs (CD11b+Ly6C+Ly6G−), and G-MDSCs (CD11b+Ly6C^{low}Ly6G+) within peritoneal cells 48 hours after thioglycolate broth infusion in control and Poly I:C-treated CSF1R^{FI/FI}-Mx1-Cre mice (n=5 mice) D) Flow cytometry analysis of M-MDSC and G-MDSC cell populations after thioglycolate infusion in control and Poly I:C-treated CSF1R^{FIFI}-Mx1-Cre mice. Peritoneal cells were collected and analyzed at 0, 2, 4, 16, 24, and 48 hours after thioglycolate infusion (n=5 mice per group).

FIGS. 3A-3B. A) Representative flow cytometry plots of CXCR2 expression on peritoneal M-MDSCs and G-MDSCs of control and Poly I:C-treated CSF1R^{FIFI}-Mx1-Cre mice (n=5 mice). Peritoneal cells were harvested 48 hrs after thioglycolate infusion (n=5 mice). B) Representative flow cytometry plots of peritoneal M-MDSCs and G-MDSCs harvested 24 hrs after thioglycolate infusion of control and Poly I:C-treated CSF1R^{FI/FI}-Mx1-Cre mice (n=5 mice). Mice were either untreated or treated with 2 mg/kg SB225002 (CXCR2 inhibitor) at time of thioglycolate infusion and 12 hrs before harvesting (n=5 mice).

FIGS. 4A-4E. A) Burden of B16-F10 melanoma tumors in control and Poly I:C-treated CSF1R^{FI/FI}-Mx1-Cre mice. CSF1R^{FI/FI}-Mx1-Cre mice received three intraperitoneal injections of Poly I:C (100 ug) and were rested for one week. 5×10\hat{ }5 B16-F10 mouse melanoma cells were then subcutaneously engrafted into flanks of control and Poly I:C-treated CSF1R^{FI/FI}-Mx1-Cre mice. Tumor size was measured every two days. Data are mean±s.e.m. (n=5 mice per group). B) Flow cytometry analysis of myeloid cells (F4/80+ macrophages, F4/80+CD206+ TAMs, M-MDSCs, G-MDSCs) from tumors. F/480+ cells are gated on CD45+ cells. CD206+ macrophages are gated on F4/80+. MDSCs are gated on CD11b+ cells. Data are mean±s.e.m. (n=5 mice per group). C) Flow cytometry analysis of MDSCs in bone marrow of tumor-bearing mice. M-MDSCs and G-MDSCs are gated on CD11b+ cells. Data are mean±s.e.m (n=5 mice per group). D) Flow cytometry analysis of MDSCs in peripheral blood from tumor-bearing mice. M-MDSCs and G-MDSCs are gated on CD11b+ cells. Data are mean±s.e.m (n=5 mice per group). E) Flow cytometry analysis of CXCR2 expression on G-MDSCs found in tumors, peripheral blood, and bone marrow of tumor-bearing mice (n=5 mice per group).

FIGS. 5A-5D. A) CSF1R^{FI/FI}-Mx1-Cre mice received three intraperitoneal injections of Poly I:C (100 ug) and were rested for one week. 5×10⁵ B16-F10 mouse melanoma cells were then subcutaneously engrafted into flanks of control and Poly I:C-treated CSF1R^{FIFI}-Mx1-Cre mice. Following the day of engraftment, mice were either untreated or treated daily with 2 mg/kg SB225002 (CXCR2 inhibitor). Burden of B16-F10 melanoma tumors in control and Poly I:C-treated CSF1R^{FIFI}-Mx1-Cre mice treated with or without SB225002. Data are mean±s.e.m. (n=5 mice per group). B) Flow cytometry analysis of M-MDSCs and G-MDSCs in tumors of mice. MDSCs are gated on CD11b+ cells. Data are mean±s.e.m. (n=5 mice per group). C) Flow cytometry analysis of M-MDSCs and G-MDSCs in bone marrow of tumor-bearing mice. MDSCs are gated on CD11b+ cells. Data are mean±s.e.m. (n=5 mice per group). D) Flow cytometry analysis of M-MDSCs and G-MDSCs in the peripheral blood of tumor-bearing mice. MDSCs are gated on CD11b+ cells. Data are mean±s.e.m. (n=5 mice per group).

FIGS. 6A-6B. A) In vivo phagocytosis assay of B16-F10 cells pre-opsonized with IgG1 or anti-CD47 (MIAP410). C57BL6 mice were infused with thioglycolate to recruit macrophages and G-MDSCs to the peritoneum. Mice were either untreated or treated with SB225002 at time of thioglycolate infusion and again 12 hrs later. 24 hours following initial thioglycolate infusion, 24 hours later, CFSE labeled B16-F10 cells pre-opsonized with IgG1 or anti-CD47 cells were injected intraperitoneally into mice. 4 hrs later, mice peritoneal cells were harvested and stained with anti-CD11b. Phagocytosis was measured by flow cytometry analysis. B) Burden of B16-F10 melanoma tumors in C57BL6 mice treated with IgG1 (100 ug every other day, 100 ug anti-CD47 every other day, 2 mg/kg SB25002 daily, or in combination). Data are mean±s.e.m (n=5 mice per group).

FIGS. 7A-7B. A) Representative bioluminescence images of ID8 ovarian tumors in C57BL6 mice at days 14 and 35. Mice were engrafted with 5×10\hat{ }6 ID8-RFP-Luc cells and were allowed to rest for one week. Mice were then treated with Cisplatin (10 mg/kg) once a week for a total of 3 doses, SB225002 (2 mg/kg) 6 days a week, a combination of both, or left untreated. B) Representative bioluminescence images of ID8 ovarian tumors in C57BL6 mice at days 14 and 35. Mice were engrafted with 5×10\hat{ }6 ID8-RFP-Luc cells and were allowed to rest for one week. Mice were first treated with three priming doses of 100 ug anti-CD47 mAb, every other day. Mice then received 300 ug doses of anti-CD47 mAb every other day. Mice received or did not receive SB225002 (2 mg/kg) 6 days a week in combination with anti-CD47 mAb treatment.

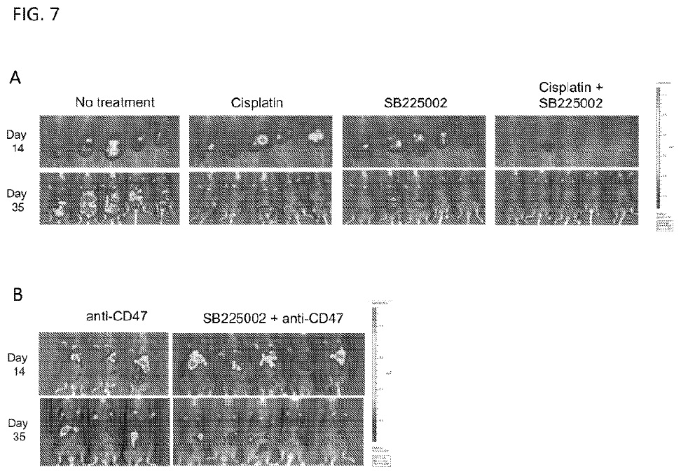

### DETAILED DESCRIPTION OF THE EMBODIMENTS

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

As used herein, compounds which are “commercially available” may be obtained from commercial sources including but not limited to Acros Organics (Pittsburgh PA), Aldrich Chemical (Milwaukee WI, including Sigma Chemical and Fluka), Apin Chemicals Ltd. (Milton Park UK), Avocado Research (Lancashire U.K.), BDH Inc. (Toronto, Canada), Bionet (Cornwall, U.K.), Chemservice Inc. (West Chester PA), Crescent Chemical Co. (Hauppauge NY), Eastman Organic Chemicals, Eastman Kodak Company (Rochester NY), Fisher Scientific Co. (Pittsburgh PA), Fisons Chemicals (Leicestershire UK), Frontier Scientific (Logan UT), ICN Biomedicals, Inc. (Costa Mesa CA), Key Organics (Cornwall U.K.), Lancaster Synthesis (Windham NH), Maybridge Chemical Co. Ltd. (Cornwall U.K.), Parish Chemical Co. (Orem UT), Pfaltz & Bauer, Inc. (Waterbury CN), Polyorganix (Houston TX), Pierce Chemical Co. (Rockford IL), Riedel de Haen AG (Hannover, Germany), Spectrum Quality Product, Inc. (New Brunswick, NJ), TCI America (Portland OR), Trans World Chemicals, Inc. (Rockville MD), Wako Chemicals USA, Inc. (Richmond VA), Novabiochem and Argonaut Technology.

Compounds can also be made by methods known to one of ordinary skill in the art. As used herein, “methods known to one of ordinary skill in the art” may be identified through various reference books and databases. Suitable reference books and treatises that detail the synthesis of reactants useful in the preparation of compounds of the present invention, or provide references to articles that describe the preparation, include for example, “Synthetic Organic Chemistry”, John Wiley & Sons, Inc., New York; S. R. Sandler et al., “Organic Functional Group Preparations,” 2nd Ed., Academic Press, New York, 1983; H. O. House, “Modern Synthetic Reactions”, 2nd Ed., W. A. Benjamin, Inc. Menlo Park, Calif. 1972; T. L. Gilchrist, “Heterocyclic Chemistry”, 2nd Ed., John Wiley & Sons, New York, 1992; J. March, “Advanced Organic Chemistry: Reactions, Mechanisms and Structure”, 4th Ed., Wiley-Interscience, New York, 1992. Specific and analogous reactants may also be identified through the indices of known chemicals prepared by the Chemical Abstract Service of the American Chemical Society, which are available in most public and university libraries, as well as through on-line databases. Chemicals that are known but not commercially available in catalogs may be prepared by custom chemical synthesis houses, where many of the standard chemical supply houses (e.g., those listed above) provide custom synthesis services.

### Definitions

CXCR2. CXCR2 belongs to the CXCR family and is the major receptor of ELR-CXC chemokines that mediate angiogenesis. It is expressed in various cell types, such as neutrophils, monocytes, eosinophils, endothelial cells, mast cells and oligodendrocytes. According to analysis of human peripheral blood leukocytes, CXCR1 and CXCR2 are expressed on neutrophils with the highest level, at an approximately equal ratio. Monocytes express CXCR2 at a higher level than CXCR1. CXCL1, CXCL2, CXCL3, CXCL5, CXCL6, CXCL7, and CXCL8 are the known ligands of CXCR2.

CXCR2 belongs to GPCR, which is a large family that contains more than 800 receptors in humans and is related to numerous human diseases. CXCR2 shares 78% sequence homology with CXCR1, and they both bind to IL-8 with similar affinity (Kd of approximately 4 nM). The seven transmembrane structure includes one N-terminus, one C-terminus, three extracellular and three cytosolic loops. The N-terminus of CXCR2 is outside the cell, whereas the C-terminus is inside the cell and contains serine and threonine residues to aid in the phosphorylation, internalization and sequestration processes of CXCR2. Several structural features are essential for ligands binding and function, such as the N-terminal segment and second extracellular loop.

MSDC inhibitor. The combination therapy of the invention encompasses the co-administration of a CD47-blocking agent and an anti-G-MSDC agent which reduces the abundance, immunosuppressive activity, or tumor recruitment of CXCR2⁺ granulocytic-myeloid derived suppressor cells. The anti-G-MDSC agent may comprise any agent that targets MDSCs, for example by depleting tumor-infiltrating populations of MDSCs, preventing MDSC recruitment to the tumor, inhibiting MDSC immunosuppressive activity, or promoting the differentiation of MDSCs to a non-suppressive state. Exemplary agents include 5-azacytidine, 5-fluorouracil, ATRA, AZD9150, CCR5 inhibitors, celecoxib, cisplatin, Cox2 inhibitors, CSF-1R inhibitors, docetaxel, entiostat, gemcitabine, HDAC inhibitors, ibrutinib, maraviroc, PDE5 inhibitors, plexidartinib, Sildenafil, STAT3 inhibitors, Tadalafil, tetrabrocinnamic acid.

CXCR2 Inhibitor. In one implementation, the anti-G-MSDC agent is an inhibitor of CXCR2. Selective and pan-specific inhibitors that act on CXCR2 are known in the art and commercially available. Examples of inhibitors useful in the methods of the disclosure include, without limitation:

| | | Selective/ | |
|---|---|---|---|
| Name | Information | Pan | IC50/Ki |
| Navaraxin | Navarixin (SCH-527123, MK-7123, PS-291822) is a | Selective | CXCR2, |
| | potent, orally bioavailable CXCR2/CXCR1 antagonist. | | IC50: 2.6 |
| | | | nM |
| SB225002 | SB225002 is a potent, and selective CXCR2 antagonist | Selective | CXCR2, |
| | | | IC50: 22 |
| | | | nM |
| SB265610 | SB265610 is a competitive antagonist at the human | Selective | |
| | CXCR2 receptor, can displace IL-8 and GROα with | | |
| | pIC50 values of 8.41 and 8.47 respectively. | | |
| AZD5069 | AZD5069 is a novel antagonist of CXCR2, which is | Selective | |
| | shown to inhibit binding of CXCL8 to CXCR2 with a | | |
| | pIC50 value of 8.8 and inhibit CXCL8 binding to CXCR1 | | |
| | with pIC50 values of 6.5. | | |
| Danirixin | Danirixin (GSK1325756) is a small molecule, non- | Selective | |
| | peptide, high affinity (IC50 for CXCL8 (IL-8) | | |
| | binding = 12.5 nM), selective, and reversible | | |
| | CXCR2 antagonist. | | |
| Reparixin | Reparixin (Repertaxin, DF 1681Y) is a potent and | Pan | CXCR2, |
| | specific inhibitor of CXCR1 with IC50 of 1 nM. | | IC50: 400 |
| | | | nM |
| SX-682 | SX-682 is an orally bioavailable small-molecule | Pan | |
| | allosteric inhibitor of CXCR1 and CXCR2 that blocks | | |
| | tumor MDSC recruitment and enhances T cell activation | | |
| | and antitumor immunity. | | |
| Elubirixin | | | |
| NSC 157449 | | | |
| MK-7123 | | | |
| QBM076 | | | |

In some embodiments a CXCR2 inhibitor is SB225002, which may be orally administered. In some embodiments a CXCR2 inhibitor is danirixin. In some embodiments a CXCR2 inhibitor is reparixin. In some embodiments a CXCR2 inhibitor is navaraxin.

The therapeutic dose may be the dose utilized in clinical trials for the specific drug, or may be, for example, at least about 0.01 μg/kg body weight, at least about 0.05 μg/kg body weight; at least about 0.1 μg/kg body weight, at least about 0.5 μg/kg body weight, at least about 1 μg/kg body weight, at least about 2.5 μg/kg body weight, at least about 5 μg/kg body weight, and not more than about 100 μg/kg body weight. The effective dose may be from about 10 μg/kg; 50 μg/kg; 100 μg/kg; 500 μg/kg, 1 mg/kg, 5 mg/k; 10 mg/kg; 25 mg/kg; 50 mg/kg; up to about 100 mg/kg in an oral administration. Effective doses for CXCR2 inhibitors other than SB225002 may be a dose that provides for an effect comparable to or better than the amounts listed above.

In certain embodiments, multiple therapeutically effective doses are administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, once every two weeks, once every three weeks, once a month, and so forth. For example, in some embodiments, an antibody is administered once every two to four weeks for an extended period of time, such as for 1, 2, 3, 4, 5, 6, 7, 8, 10, 15, 24 months, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted herein below.

In certain embodiments, multiple therapeutically effective doses are administered according to a daily dosing regimen, or intermittently. For example, a therapeutically effective dose can be administered, one day a week, two days a week, three days a week, four days a week, or five days a week, and so forth. By “intermittent” administration is intended the therapeutically effective dose can be administered, for example, every other day, every two days, every three days, once a week, once every two weeks, once every three weeks, once a month, and so forth. For example, in some embodiments, an antibody is administered once every two to four weeks for an extended period of time, such as for 1, 2, 3, 4, 5, 6, 7, 8, 10, 15, 24 months, and so forth. By “twice-weekly” or “two times per week” is intended that two therapeutically effective doses of the agent in question is administered to the subject within a 7 day period, beginning on day 1 of the first week of administration, with a minimum of 72 hours, between doses and a maximum of 96 hours between doses. By “thrice weekly” or “three times per week” is intended that three therapeutically effective doses are administered to the subject within a 7 day period, allowing for a minimum of 48 hours between doses and a maximum of 72 hours between doses. For purposes of the present invention, this type of dosing is referred to as “intermittent” therapy. In accordance with the methods of the present invention, a subject can receive intermittent therapy for one or more weekly or monthly cycles until the desired therapeutic response is achieved. The agents can be administered by any acceptable route of administration as noted herein below.

Alternative inhibitors of CXCR2 that find use in the methods of the disclosure include, for example, antibodies. Antibodies known in the art include TAHX2, a mAb specific for human CXCR2 (hCXCR2), where the humanized version of TAHX2 (HAHX2) may be used. High affinity antibodies are described by Shi et al. (2021) Nature Communications 12, Article number: 2547.

Anti-CD47 agent. CD47 is a broadly expressed transmembrane glycoprotein with a single Ig-like domain and five membrane spanning regions, which functions as a cellular ligand for SIRPα with binding mediated through the NH2-terminal V-like domain of SIRPα. SIRPα is expressed primarily on myeloid cells, including macrophages, granulocytes, myeloid dendritic cells (DCs), mast cells, and their precursors, including hematopoietic stem cells.

As used herein, the term “anti-CD47 agent” or “agent that provides for CD47 blockade” refers to any agent that reduces the binding of CD47 (e.g., on a target cell) to SIRPα (e.g., on a phagocytic cell). Non-limiting examples of suitable anti-CD47 reagents include SIRPα reagents, which include without limitation high affinity SIRPα polypeptides, anti-SIRPα antibodies; and CD47 agents, which include soluble CD47 polypeptides, and anti-CD47 antibodies or antibody fragments.

In some embodiments, a suitable anti-CD47 agent (e.g. an anti-CD47 antibody, a SIRPα polypeptide, etc.) specifically binds CD47 to reduce the binding of CD47 to SIRPα.

In some embodiments, a suitable anti-SIRPα agent (e.g., an anti-SIRPα antibody, a soluble CD47 polypeptide, etc.) specifically binds SIRPα to reduce the binding of CD47 to SIRPα.

A suitable agent that binds SIRPα does not activate SIRPα (e.g., in the SIRPα-expressing phagocytic cell). In some embodiments, the anti-CD47 agent does not activate CD47 upon binding. When CD47 is activated, a process akin to apoptosis (i.e., programmed cell death) may occur (Manna and Frazier, Cancer Research, 64, 1026-1036 Feb. 1 2004). Thus, in some embodiments, the anti-CD47 agent does not directly induce cell death of a CD47-expressing cell, i.e. does not directly induce apoptosis.

The efficacy of a suitable agent can be assessed by assaying the agent. In an exemplary assay, target cells are incubated in the presence or absence of the candidate agent and in the presence of an effector cell, e.g. a macrophage or other phagocytic cell. An agent for use in the methods of the invention will up-regulate phagocytosis by at least 5% (e.g., at least 10%, at least 20%, at least 30%, at least 40%, at least 50%, at least 60%, at least 70%, at least 80%, at least 90%, at least 100%, at least 120%, at least 140%, at least 160%, at least 180%, at least 200%, at least 500%, at least 1000%) compared to phagocytosis in the absence of the agent.

In some embodiments a primer agent is administered prior to administering a therapeutically effective dose of an anti-CD47 agent to the individual. Suitable primer agents include an erythropoiesis-stimulating agent (ESA), and/or a subtherapeutic dose of an anti-CD47 agent. Following administration of the priming agent, and allowing a period of time effective for an increase in reticulocyte production, a therapeutic dose of an anti-CD47 agent is administered. Administration may be made in accordance with the methods described in U.S. Pat. Nos. 9,623,079; 10,301,387; and 11,136,391, each herein specifically incorporated by reference.

Anti-CD47 agents useful in the methods of the disclosure and currently in clinical trials include, for example:

| | |
|---|---|
| Hu5F9-G4/Magrolimab (Forty Seven Inc/Gilead) | CD47 mAb |
| CC-90002 (Celgene) | CD47 mAb |
| IBI188 (Innovent Biologics) | CD47 mAb |
| TTI-621 (Trillium Therapeutics) | SIRPα-Ig fusion |
| | protein |
| TTI-622 (trillium) | SIRPα-IgG4 fusion |
| | protein |
| HCB-101 (Hanchorbio) | SIRPα-IgG fusion |
| SRF231 (Surface Oncology) | CD47 mAb |
| AO-176 (Arch Oncology) | CD47 mAb |
| Evorpacept (ALX Therapeutics) | SIRPα-Fc* fusion |
| | protein |
| CC-95251 (Celgene) | SIRPα mAb |
| TG-1801 (TG Therapeutics) | CD47/CD19 bsAb |
| BI765063/OSE-172 (Boehringer Ingelheim/OSE) | SIRPα mAb |
| SGN CD47M (Seattle Genetics) | mAb-drug conjugate |
| IBI-322 (Innovent Biologics) | CD47/PD-L1 bsAb |
| HX-009 (Waterstone Hanxbio) | PD-1/CD47 bsAb |
| IMC-002 (ImmuneOncia) | CD47 mAb |
| IMC-002 (ImmuneOncia Therapeutics LLC) | anti-CD47 mAb |
| Lemzoparlimab (I-Mab Bio-Tech (Tianjin) Co., Ltd) | anti-CD47 mAb |
| Letaplimab (Innovent Biologics Inc) | anti-CD47 mAb |
| Ligufalimab (Akeso Biopharma Inc) | anti-CD47 mAb |
| MIL-95 (KeyMed Biosciences Co Ltd) | anti-CD47 mAb |
| SHR-1603 (Jiangsu Hengrui Medicine Co Ltd) | anti-CD47 mAb |
| ZL-1201 (Zai Lab Limited) | anti-CD47 mAb |
| BAT-7104 (Bio-Thera Solutions Ltd) | bispecific antibody |
| HX-009 (HanX Biopharmaceuticals Inc) | bispecific antibody |
| IMM-0306 (ImmuneOnco Biopharm Co Ltd) | bispecific antibody |
| JMT-601 (Shanghai JMT-Bio Inc) | bispecific antibody |
| SG-12473 (Hangzhou Sumgen Biotechnology Co | bispecific antibody |
| Ltd) | |
| XL-114 (Exelixis Inc) | bispecific antibody |
| IMM-01 (ImmuneOnco Biopharm Co Ltd) | SIRPα/Fc fusion |
| | protein |
| IMM-01 (ImmuneOnco Biopharm Co Ltd) | SIRPα/Fc fusion |
| | protein |
| RRx-001 (EpiCentrx Inc) | small-molecule |
| | inhibitor |
| IMM2902 | bispecific antibody |

A “therapeutically effective dose” or “therapeutic dose” of an anti-CD47 agent is an amount that is sufficient to palliate, ameliorate, stabilize, reverse, prevent, slow or delay the progression of the disease state by increasing phagocytosis of a target cell. A therapeutically effective dose of an anti-CD47 agent reduces the binding of CD47 on a target cell to SIRPα on a phagocytic cell at an effective dose for increasing the phagocytosis of the target cell.

In some embodiments, a therapeutically effective dose leads to sustained serum levels of anti-CD47 agent of about 40 μg/ml or more, e.g, about 50 μg/ml or more, about 60 μg/ml or more, about 75 μg/ml or more, about 100 μg/ml or more, about 125 μg/ml or more, or about 150 μg/ml or more). In some embodiments, a therapeutically effective dose leads to sustained serum levels of anti-CD47 agent that range from about 40 μg/ml to about 300 μg/ml, e.g, from about 40 μg/ml to about 250 μg/ml, from about 40 μg/ml to about 200 μg/ml, from about 40 μg/ml to about 150 μg/ml, from about 40 μg/ml to about 100 μg/ml, from about 50 μg/ml to about 300 μg/ml, from about 50 μg/ml to about 250 μg/ml, from about 50 μg/ml to about 200 μg/ml, from about 50 μg/ml to about 150 μg/ml, from about 75 μg/ml to about 300 μg/ml from about 75 μg/ml to about 250 μg/ml, from about 75 μg/ml to about 200 μg/ml, from about 75 μg/ml to about 150 μg/ml, from about 100 μg/ml to about 300 μg/ml, from about 100 μg/ml to about 250 μg/ml, or from about 100 μg/ml to about 200 μg/ml). In some embodiments, a therapeutically effective dose for treating solid tumors leads to sustained serum levels of anti-CD47 agent of about 100 μg/ml or more, e.g., sustained serum levels that range from about 100 μg/ml to about 200 μg/ml.

A therapeutically effective dose of an anti-CD47 agent can depend on the specific agent used, but is usually about 5 mg/kg body weight or more, e.g., about 8 mg/kg or more, about 10 mg/kg or more, about 15 mg/kg or more, about 20 mg/kg or more, about 25 mg/kg or more, about 30 mg/kg or more, about 35 mg/kg or more, about 40 mg/kg or more, about 45 mg/kg or more, about 50 mg/kg; or from about 10 mg/kg to about 50 mg/kg (e.g., from about 20 mg/kg to about 35 mg/kg, or from about 25 mg/kg to about 30 mg/kg). The dose required to achieve and/or maintain a particular serum level is proportional to the amount of time between doses and inversely proportional to the number of doses administered. Thus, as the frequency of dosing increases, the required dose decreases. The optimization of dosing strategies will be readily understood and practiced by one of ordinary skill in the art.

In some embodiments, the anti-CD47 agent is Magrolimab (5F9-G4). An exemplary dosing regimen may be, for example, intravenous administration of an initial 1 mg/kg priming dose to mitigate on target anemia. An intrapatient dose escalation regimen up to 30 mg/kg is then administered through Cycle 1, 30 mg/kg weekly dosing in Cycle 2, with 30 mg/kg Q2W dosing occurring in Cycle 3 and beyond.

SIRPα reagent. A SIRPα reagent comprises the portion of SIRPα that is sufficient to bind CD47 at a recognizable affinity, which normally lies between the signal sequence and the transmembrane domain, or a fragment thereof that retains the binding activity. A suitable SIRPα reagent reduces (e.g., blocks, prevents, etc.) the interaction between the native proteins SIRPα and CD47. The SIRPα reagent will usually comprise at least the d1 domain of SIRPα.

In some embodiments, a subject anti-CD47 agent is a “high affinity SIRPα reagent”, which includes SIRPα-derived polypeptides and analogs thereof (e.g., CV1-hIgG4, and CV1 monomer). High affinity SIRPα reagents are described in U.S. Pat. No. 9,944,911; which is hereby specifically incorporated by reference. High affinity SIRPα reagents are variants of the native SIRPα protein. The amino acid changes that provide for increased affinity are localized in the d1 domain, and thus high affinity SIRPα reagents comprise a d1 domain of human SIRPα, with at least one amino acid change relative to the wild-type sequence within the d1 domain. Such a high affinity SIRPα reagent optionally comprises additional amino acid sequences, for example antibody Fc sequences; portions of the wild-type human SIRPα protein other than the d1 domain, including without limitation residues 150 to 374 of the native protein or fragments thereof, usually fragments contiguous with the d1 domain; and the like. High affinity SIRPα reagents may be monomeric or multimeric, i.e. dimer, trimer, tetramer, etc. In some embodiments, a high affinity SIRPα reagent is soluble, where the polypeptide lacks the SIRPα transmembrane domain and comprises at least one amino acid change relative to the wild-type SIRPα sequence, and wherein the amino acid change increases the affinity of the SIRPα polypeptide binding to CD47, for example by decreasing the off-rate by at least 10-fold, at least 20-fold, at least 50-fold, at least 100-fold, at least 500-fold, or more.

Optionally the SIRPα reagent is a fusion protein, e.g., fused in frame with a second polypeptide. In some embodiments, the second polypeptide is capable of increasing the size of the fusion protein, e.g., so that the fusion protein will not be cleared from the circulation rapidly. In some embodiments, the second polypeptide is part or whole of an immunoglobulin Fc region. The Fc region aids in phagocytosis by providing an “eat me” signal, which enhances the block of the “don't eat me” signal provided by the high affinity SIRPα reagent. In other embodiments, the second polypeptide is any suitable polypeptide that is substantially similar to Fc, e.g., providing increased size, multimerization domains, and/or additional binding or interaction with Ig molecules.

Anti-CD47 antibodies. In some embodiments, a subject anti-CD47 agent is an antibody that specifically binds CD47 (i.e., an anti-CD47 antibody) and reduces the interaction between CD47 on one cell (e.g., an infected cell) and SIRPα on another cell (e.g., a phagocytic cell). In some embodiments, a suitable anti-CD47 antibody does not activate CD47 upon binding. Some anti-CD47 antibodies do not reduce the binding of CD47 to SIRPα (and are therefore not considered to be an “anti-CD47 agent” herein) and such an antibody can be referred to as a “non-blocking anti-CD47 antibody.” A suitable anti-CD47 antibody that is an “anti-CD47 agent” can be referred to as a “CD47-blocking antibody”. Non-limiting examples of suitable antibodies include clones B6H12, 5F9, 8B6, and C3 (for example as described in International Patent Publication WO 2011/143624, herein specifically incorporated by reference). Suitable anti-CD47 antibodies include fully human, humanized or chimeric versions of such antibodies. Humanized antibodies, e.g. Magrolimab (hu5F9-G4) are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

In some embodiments an anti-CD47 antibody comprises a human IgG Fc region, e.g. an IgG1, IgG2a, IgG2b, IgG3, IgG4 constant region. In a preferred embodiment the IgG Fc region is an IgG4 constant region. The IgG4 hinge may be stabilized by the amino acid substitution S241P (see Angal et al. (1993) Mol. Immunol. 30(1):105-108, herein specifically incorporated by reference).

Anti-SIRPα antibodies. In some embodiments, a subject anti-CD47 agent is an antibody that specifically binds SIRPα (i.e., an anti-SIRPα antibody) and reduces the interaction between CD47 on one cell (e.g., an infected cell) and SIRPα on another cell (e.g., a phagocytic cell). Suitable anti-SIRPα antibodies can bind SIRPα without activating or stimulating signaling through SIRPα because activation of SIRPα would inhibit phagocytosis. Instead, suitable anti-SIRPα antibodies facilitate the preferential phagocytosis of inflicted cells over normal cells. Those cells that express higher levels of CD47 (e.g., infected cells) relative to other cells (non-infected cells) will be preferentially phagocytosed. Thus, a suitable anti-SIRPα antibody specifically binds SIRPα (without activating/stimulating enough of a signaling response to inhibit phagocytosis) and blocks an interaction between SIRPα and CD47. Suitable anti-SIRPα antibodies include fully human, humanized or chimeric versions of such antibodies. Humanized antibodies are especially useful for in vivo applications in humans due to their low antigenicity. Similarly caninized, felinized, etc. antibodies are especially useful for applications in dogs, cats, and other species respectively. Antibodies of interest include humanized antibodies, or caninized, felinized, equinized, bovinized, porcinized, etc., antibodies, and variants thereof.

Soluble CD47 polypeptides. In some embodiments, a subject anti-CD47 agent is a soluble CD47 polypeptide that specifically binds SIRPα and reduces the interaction between CD47 on one cell (e.g., an infected cell) and SIRPα on another cell (e.g., a phagocytic cell). A suitable soluble CD47 polypeptide can bind SIRPα without activating or stimulating signaling through SIRPα because activation of SIRPα would inhibit phagocytosis. Instead, suitable soluble CD47 polypeptides facilitate the preferential phagocytosis of infected cells over non-infected cells. Those cells that express higher levels of CD47 (e.g., infected cells) relative to normal, non-target cells (normal cells) will be preferentially phagocytosed. Thus, a suitable soluble CD47 polypeptide specifically binds SIRPα without activating/stimulating enough of a signaling response to inhibit phagocytosis.

In some cases, a suitable soluble CD47 polypeptide can be a fusion protein (for example as structurally described in US Patent Publication US20100239579, herein specifically incorporated by reference). However, only fusion proteins that do not activate/stimulate SIRPα are suitable for the methods provided herein. Suitable soluble CD47 polypeptides also include any peptide or peptide fragment comprising variant or naturally existing CD47 sequences (e.g., extracellular domain sequences or extracellular domain variants) that can specifically bind SIRPα and inhibit the interaction between CD47 and SIRPα without stimulating enough SIRPα activity to inhibit phagocytosis.

In certain embodiments, soluble CD47 polypeptide comprises the extracellular domain of CD47, including the signal peptide, such that the extracellular portion of CD47 is typically 142 amino acids in length. The soluble CD47 polypeptides described herein also include CD47 extracellular domain variants that comprise an amino acid sequence at least 65%-75%, 75%-80%, 80-85%, 85%-90%, or 95%-99% (or any percent identity not specifically enumerated between 65% to 100%), which variants retain the capability to bind to SIRPα without stimulating SIRPα signaling.

In certain embodiments, the signal peptide amino acid sequence may be substituted with a signal peptide amino acid sequence that is derived from another polypeptide (e.g., for example, an immunoglobulin or CTLA4). For example, unlike full-length CD47, which is a cell surface polypeptide that traverses the outer cell membrane, the soluble CD47 polypeptides are secreted; accordingly, a polynucleotide encoding a soluble CD47 polypeptide may include a nucleotide sequence encoding a signal peptide that is associated with a polypeptide that is normally secreted from a cell.

In other embodiments, the soluble CD47 polypeptide comprises an extracellular domain of CD47 that lacks the signal peptide. As described herein, signal peptides are not exposed on the cell surface of a secreted or transmembrane protein because either the signal peptide is cleaved during translocation of the protein or the signal peptide remains anchored in the outer cell membrane (such a peptide is also called a signal anchor). The signal peptide sequence of CD47 is believed to be cleaved from the precursor CD47 polypeptide in vivo.

In other embodiments, a soluble CD47 polypeptide comprises a CD47 extracellular domain variant. Such a soluble CD47 polypeptide retains the capability to bind to SIRPα without stimulating SIRPα signaling. The CD47 extracellular domain variant may have an amino acid sequence that is at least 65%-75%, 75%-80%, 80-85%, 85%-90%, or 95%-99% identical (which includes any percent identity between any one of the described ranges) to the native CD47 sequence.

As used herein, “antibody” includes reference to an immunoglobulin molecule immunologically reactive with a particular antigen, and includes both polyclonal and monoclonal antibodies. The term also includes genetically engineered forms such as chimeric antibodies (e.g., humanized murine antibodies) and heteroconjugate antibodies. The term “antibody” also includes antigen binding forms of antibodies, including fragments with antigen-binding capability (e.g., Fab', F(ab')₂, Fab, Fv and rIgG. The term also refers to recombinant single chain Fv fragments (scFv). The term antibody also includes bivalent or bispecific molecules, diabodies, triabodies, and tetrabodies.

Selection of antibodies may be based on a variety of criteria, including selectivity, affinity, cytotoxicity, etc. The phrase “specifically (or selectively) binds” to an antibody or “specifically (or selectively) immunoreactive with,” when referring to a protein or peptide, refers to a binding reaction that is determinative of the presence of the protein, in a heterogeneous population of proteins and other biologics. Thus, under designated immunoassay conditions, the specified antibodies bind to a particular protein sequences at least two times the background and more typically more than 10 to 100 times background. In general, antibodies of the present invention bind antigens on the surface of target cells in the presence of effector cells (such as natural killer cells or macrophages). Fc receptors on effector cells recognize bound antibodies.

An antibody that is immunologically reactive with a particular antigen can be generated by recombinant methods such as selection of libraries of recombinant antibodies in phage or similar vectors, or by immunizing an animal with the antigen or with DNA encoding the antigen. Methods of preparing polyclonal antibodies are known to the skilled artisan. The antibodies may, alternatively, be monoclonal antibodies. Monoclonal antibodies may be prepared using hybridoma methods. In a hybridoma method, an appropriate host animal is typically immunized with an immunizing agent to elicit lymphocytes that produce or are capable of producing antibodies that will specifically bind to the immunizing agent. Alternatively, the lymphocytes may be immunized in vitro. The lymphocytes are then fused with an immortalized cell line using a suitable fusing agent, such as polyethylene glycol, to form a hybridoma cell.

Human antibodies can be produced using various techniques known in the art, including phage display libraries. Similarly, human antibodies can be made by introducing of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire.

Antibodies also exist as a number of well-characterized fragments produced by digestion with various peptidases. Thus pepsin digests an antibody below the disulfide linkages in the hinge region to produce F(ab)'₂, a dimer of Fab which itself is a light chain joined to V_{H}-C_{H1} by a disulfide bond. The F(ab)'₂ may be reduced under mild conditions to break the disulfide linkage in the hinge region, thereby converting the F(ab)'₂ dimer into an Fab' monomer. The Fab' monomer is essentially Fab with part of the hinge region. While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries.

A “humanized antibody” is an immunoglobulin molecule which contains minimal sequence derived from non-human immunoglobulin. Humanized antibodies include human immunoglobulins (recipient antibody) in which residues from a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, a humanized antibody will comprise substantially all of at least one, and typically two, variable domains, in which all or substantially all of the CDR regions correspond to those of a non-human immunoglobulin and all or substantially all of the framework (FR) regions are those of a human immunoglobulin consensus sequence. The humanized antibody optimally also will comprise at least a portion of an immunoglobulin constant region (Fc), typically that of a human immunoglobulin.

Antibodies of interest may be tested for their ability to induce ADCC (antibody-dependent cellular cytotoxicity) or ADCP (antibody dependent cellular phagocytosis). Antibody-associated ADCC activity can be monitored and quantified through detection of either the release of label or lactate dehydrogenase from the lysed cells, or detection of reduced target cell viability (e.g. annexin assay). Assays for apoptosis may be performed by terminal deoxynucleotidyl transferase-mediated digoxigenin-11-dUTP nick end labeling (TUNEL) assay (Lazebnik et al., Nature: 371, 346 (1994). Cytotoxicity may also be detected directly by detection kits known in the art, such as Cytotoxicity Detection Kit from Roche Applied Science (Indianapolis, Ind.).

A “patient” for the purposes of the present invention includes both humans and other animals, particularly mammals, including pet and laboratory animals, e.g. mice, rats, rabbits, etc. Thus the methods are applicable to both human therapy and veterinary applications. In one embodiment the patient is a mammal, preferably a primate. In other embodiments the patient is human.

The terms “subject,” “individual,” and “patient” are used interchangeably herein to refer to a mammal being assessed for treatment and/or being treated. In an embodiment, the mammal is a human. The terms “subject,” “individual,” and “patient” encompass, without limitation, individuals having cancer. Subjects may be human, but also include other mammals, particularly those mammals useful as laboratory models for human disease, e.g. mouse, rat, etc.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. Many types of cancers are known to those of skill in the art, including solid tumors such as carcinomas, sarcomas, glioblastomas, melanomas, lymphomas, myelomas, etc., and circulating cancers such as leukemias. Examples of cancer include but are not limited to, ovarian cancer, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer.

Selection of patients and tumors for CD47 blockade combination therapy with a CXCR2 inhibitor may include determining the presence of tumor-associate macrophages (TAM) and in particular CXCR2⁺ granulocyte myeloid derived suppressor cells (G-MDSC) in the tumor microenvironment, where the presence can be indicative that the individual will be responsive. In some implementations, the presence of circulating or tumor MDSCs is assessed by the process of collecting a sample (e.g. blood, or tumor biopsy) wherein the sample is assessed for the presence of MSDCs, for example, MSDC's in excess of normal baseline abundance of comparable healthy tissues. MSDC's may be assessed by various biomarkers known in the art for example, CD11b+, CD33+, HLA-DRlow/− and negative for lineage-specific antigen (Lin−), for example CD11b+CD33+HLA-DR−/CD14+CD15− for M-MDSC and CD11b+CD33+HLA-DR−/CD14-CD15+ for PMN-MDSCs. Additional markers of MDSC Identity include, for example, CCR2, CXCR4, CD11b, CD13, CD14, CD15, CD16, CD33, CD34, CD38, CD39, CD45, CD62L, CD66b, CD68, CD73, CD80, CD83, CD86, CD115, CXCR1, and Lin. Thus, the anti-tumor efficacy of tumors that are responsive to these therapies is enhanced and the therapeutic response of tumors that are not responsive is enabled. A combination of CD47 blockade, with CXCR2 inhibitors as described herein is given to patients with tumor subtypes that are responsive to these therapies.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

The types of cancer that can be treated using the subject methods of the present invention include but are not limited to adrenal cortical cancer, anal cancer, aplastic anemia, bile duct cancer, bladder cancer, bone cancer, bone metastasis, brain cancers, central nervous system (CNS) cancers, peripheral nervous system (PNS) cancers, breast cancer, cervical cancer, childhood Non-Hodgkin's lymphoma, colon and rectum cancer, endometrial cancer, esophagus cancer, Ewing's family of tumors (e.g. Ewing's sarcoma), eye cancer, gallbladder cancer, gastrointestinal carcinoid tumors, gastrointestinal stromal tumors, gestational trophoblastic disease, hairy cell leukemia, Hodgkin's lymphoma, Kaposi's sarcoma, kidney cancer, laryngeal and hypopharyngeal cancer, acute lymphocytic leukemia, acute myeloid leukemia, children's leukemia, chronic lymphocytic leukemia, chronic myeloid leukemia, liver cancer, lung cancer, lung carcinoid tumors, Non-Hodgkin's lymphoma, male breast cancer, malignant mesothelioma, multiple myeloma, myelodysplastic syndrome, myeloproliferative disorders, nasal cavity and paranasal cancer, nasopharyngeal cancer, neuroblastoma, oral cavity and oropharyngeal cancer, osteosarcoma, ovarian cancer, pancreatic cancer, penile cancer, pituitary tumor, prostate cancer, retinoblastoma, rhabdomyosarcoma, salivary gland cancer, sarcomas, melanoma skin cancer, non-melanoma skin cancers, stomach cancer, testicular cancer, thymus cancer, thyroid cancer, uterine cancer (e.g. uterine sarcoma), transitional cell carcinoma, vaginal cancer, vulvar cancer, mesothelioma, squamous cell or epidermoid carcinoma, bronchial adenoma, choriocarinoma, head and neck cancers, teratocarcinoma, or Waldenstrom's macroglobulinemia.

In some embodiments the cancer is a solid cancer, e.g. lymphoma, carcinoma, sarcoma, melanoma, etc. In some embodiments the solid cancer is melanoma. In some embodiments the solid cancer is ovarian cancer. In some embodiments the solid cancer is a carcinoma.

In other embodiments the methods of the invention are utilized in the treatment of inflammation associated with myeloid-derived suppressor cells. In some embodiments, inflammation associated with myeloid derived suppressor cells is peritonitis. Peritonitis is inflammation of the localized or generalized peritoneum, the lining of the inner wall of the abdomen and cover of the abdominal organs. Symptoms may include severe pain, swelling of the abdomen, fever, or weight loss. One part or the entire abdomen may be tender. Complications may include shock and acute respiratory distress syndrome. Causes include perforation of the intestinal tract, pancreatitis, pelvic inflammatory disease, stomach ulcer, cirrhosis, or a ruptured appendix. Risk factors include ascites (the abnormal build-up of fluid in the abdomen) and peritoneal dialysis. Diagnosis is generally based on examination, blood tests, and medical imaging. Treatment often includes antibiotics, intravenous fluids, pain medication, and surgery. Other measures may include a nasogastric tube or blood transfusion.

Perforation of part of the gastrointestinal tract is the most common cause of peritonitis. Examples include perforation of the distal esophagus (Boerhaave syndrome), of the stomach (peptic ulcer, gastric carcinoma), of the duodenum (peptic ulcer), of the remaining intestine (e.g., appendicitis, diverticulitis, Meckel diverticulum, inflammatory bowel disease (IBD), intestinal infarction, intestinal strangulation, colorectal carcinoma, meconium peritonitis), or of the gallbladder (cholecystitis). Other possible reasons for perforation include abdominal trauma, ingestion of a sharp foreign body (such as a fish bone, toothpick or glass shard), perforation by an endoscope or catheter, and anastomotic leakage. The latter occurrence is particularly difficult to diagnose early, as abdominal pain and ileus paralyticus are considered normal in people who have just undergone abdominal surgery. In most cases of perforation of a hollow viscus, mixed bacteria are isolated; the most common agents include Gram-negative bacilli (e.g., Escherichia coli) and anaerobic bacteria (e.g., Bacteroides fragilis). Fecal peritonitis results from the presence of feces in the peritoneal cavity. It can result from abdominal trauma and occurs if the large bowel is perforated during surgery.

Disruption of the peritoneum, even in the absence of perforation of a hollow viscus, may also cause infection simply by letting micro-organisms into the peritoneal cavity. Examples include trauma, surgical wound, continuous ambulatory peritoneal dialysis, and intra-peritoneal chemotherapy. Again, in most cases, mixed bacteria are isolated; the most common agents include cutaneous species such as Staphylococcus aureus, and coagulase-negative staphylococci, but many others are possible, including fungi such as Candida.

Dosage and frequency may vary depending on the half-life of the agent in the patient. It will be understood by one of skill in the art that such guidelines will be adjusted for the molecular weight of the active agent, the clearance from the blood, the mode of administration, and other pharmacokinetic parameters. The dosage may also be varied for localized administration, e.g. intranasal, inhalation, etc., or for systemic administration, e.g. i.m., i.p., i.v., oral, and the like.

An active agent can be administered by any suitable means, including topical, oral, parenteral, intrapulmonary, and intranasal. Parenteral infusions include intramuscular, intravenous (bolus or slow drip), intraarterial, intraperitoneal, intrathecal or subcutaneous administration. An agent can be administered in any manner which is medically acceptable. This may include injections, by parenteral routes such as intravenous, intravascular, intraarterial, subcutaneous, intramuscular, intratumor, intraperitoneal, intraventricular, intraepidural, or others as well as oral, nasal, ophthalmic, rectal, or topical. Sustained release administration is also specifically included in the disclosure, by such means as depot injections or erodible implants.

As noted above, an agent can be formulated with an a pharmaceutically acceptable carrier (one or more organic or inorganic ingredients, natural or synthetic, with which a subject agent is combined to facilitate its application). A suitable carrier includes sterile saline although other aqueous and non-aqueous isotonic sterile solutions and sterile suspensions known to be pharmaceutically acceptable are known to those of ordinary skill in the art. An “effective amount” refers to that amount which is capable of ameliorating or delaying progression of the diseased, degenerative or damaged condition. An effective amount can be determined on an individual basis and will be based, in part, on consideration of the symptoms to be treated and results sought. An effective amount can be determined by one of ordinary skill in the art employing such factors and using no more than routine experimentation.

An agent can be administered as a pharmaceutical composition comprising a pharmaceutically acceptable excipient. The preferred form depends on the intended mode of administration and therapeutic application. The compositions can also include, depending on the formulation desired, pharmaceutically-acceptable, non-toxic carriers or diluents, which are defined as vehicles commonly used to formulate pharmaceutical compositions for animal or human administration. The diluent is selected so as not to affect the biological activity of the combination. Examples of such diluents are distilled water, physiological phosphate-buffered saline, Ringer's solutions, dextrose solution, and Hank's solution. In addition, the pharmaceutical composition or formulation may also include other carriers, adjuvants, or nontoxic, nontherapeutic, nonimmunogenic stabilizers and the like.

The active agents of the invention and/or the compounds administered therewith are incorporated into a variety of formulations for therapeutic administration. In one aspect, the agents are formulated into pharmaceutical compositions by combination with appropriate, pharmaceutically acceptable carriers or diluents, and are formulated into preparations in solid, semi-solid, liquid or gaseous forms, such as tablets, capsules, powders, granules, ointments, solutions, suppositories, injections, inhalants, gels, microspheres, and aerosols. As such, administration of the active agents and/or other compounds can be achieved in various ways, usually by oral administration. The active agents and/or other compounds may be systemic after administration or may be localized by virtue of the formulation, or by the use of an implant that acts to retain the active dose at the site of implantation.

In pharmaceutical dosage forms, the active agents and/or other compounds may be administered in the form of their pharmaceutically acceptable salts, or they may also be used alone or in appropriate association, as well as in combination with other pharmaceutically active compounds. The agents may be combined, as previously described, to provide a cocktail of activities. The following methods and excipients are exemplary and are not to be construed as limiting the invention.

The term “sample” with respect to a patient encompasses blood and other liquid samples of biological origin, solid tissue samples such as a biopsy specimen or tissue cultures or cells derived therefrom and the progeny thereof. The definition also includes samples that have been manipulated in any way after their procurement, such as by treatment with reagents; washed; or enrichment for certain cell populations, such as cancer cells. The definition also includes sample that have been enriched for particular types of molecules, e.g., nucleic acids, polypeptides, etc. The term “biological sample” encompasses a clinical sample, and also includes tissue obtained by surgical resection, tissue obtained by biopsy, cells in culture, cell supernatants, cell lysates, tissue samples, organs, bone marrow, blood, plasma, serum, and the like. A “biological sample” includes a sample obtained from a patient's cancer cell, e.g., a sample comprising polynucleotides and/or polypeptides that is obtained from a patient's cancer cell (e.g., a cell lysate or other cell extract comprising polynucleotides and/or polypeptides); and a sample comprising cancer cells from a patient. A biological sample comprising a cancer cell from a patient can also include non-cancerous cells.

The term “diagnosis” is used herein to refer to the identification of a molecular or pathological state, disease or condition, such as the identification of a molecular subtype of breast cancer, prostate cancer, or other type of cancer.

The term “prognosis” is used herein to refer to the prediction of the likelihood of cancer-attributable death or progression, including recurrence, metastatic spread, and drug resistance, of a neoplastic disease, such as ovarian cancer. The term “prediction” is used herein to refer to the act of foretelling or estimating, based on observation, experience, or scientific reasoning. In one example, a physician may predict the likelihood that a patient will survive, following surgical removal of a primary tumor and/or chemotherapy for a certain period of time without cancer recurrence.

As used herein, the terms “treatment,” “treating,” and the like, refer to administering an agent, or carrying out a procedure, for the purposes of obtaining an effect. The effect may be prophylactic in terms of completely or partially preventing a disease or symptom thereof and/or may be therapeutic in terms of effecting a partial or complete cure for a disease and/or symptoms of the disease. “Treatment,” as used herein, may include treatment of a tumor in a mammal, particularly in a human, and includes: (a) preventing the disease or a symptom of a disease from occurring in a subject which may be predisposed to the disease but has not yet been diagnosed as having it (e.g., including diseases that may be associated with or caused by a primary disease; (b) inhibiting the disease, i.e., arresting its development; and (c) relieving the disease, i.e., causing regression of the disease.

Treating may refer to any indicia of success in the treatment or amelioration or prevention of a cancer, including any objective or subjective parameter such as abatement; remission; diminishing of symptoms or making the disease condition more tolerable to the patient; slowing in the rate of degeneration or decline; or making the final point of degeneration less debilitating. The treatment or amelioration of symptoms can be based on objective or subjective parameters; including the results of an examination by a physician. Accordingly, the term “treating” includes the administration of the compounds or agents of the present invention to prevent or delay, to alleviate, or to arrest or inhibit development of the symptoms or conditions associated with cancer or other diseases. The term “therapeutic effect” refers to the reduction, elimination, or prevention of the disease, symptoms of the disease, or side effects of the disease in the subject.

“In combination with”, “combination therapy” and “combination products” refers to the concurrent administration to a patient of the agents described herein. When administered in combination, each component can be administered at the same time or sequentially in any order at different points in time. Thus, each component can be administered separately but sufficiently closely in time so as to provide the desired therapeutic effect.

“Concomitant administration” of active agents in the methods of the invention means administration with the reagents at such time that the agents will have a therapeutic effect at the same time. Such concomitant administration may involve concurrent (i.e. at the same time), prior, or subsequent administration of the agents. A person of ordinary skill in the art would have no difficulty determining the appropriate timing, sequence and dosages of administration for particular drugs and compositions of the present invention.

“In combination with”, “combination therapy” and “combination products” refer, in certain embodiments, to the concurrent administration to a patient of the engineered proteins and cells described herein in combination with additional therapies, e.g. surgery, radiation, chemotherapy, and the like. When administered in combination, each component can be administered at the same time or sequentially in any order at different points in time. Thus, each component can be administered separately but sufficiently closely in time so as to provide the desired therapeutic effect.

“Concomitant administration” means administration of one or more components, such as engineered proteins and cells, known therapeutic agents, etc. at such time that the combination will have a therapeutic effect. Such concomitant administration may involve concurrent (i.e. at the same time), prior, or subsequent administration of components. A person of ordinary skill in the art would have no difficulty determining the appropriate timing, sequence and dosages of administration.

The use of the term “in combination” does not restrict the order in which prophylactic and/or therapeutic agents are administered to a subject with a disorder. A first prophylactic or therapeutic agent can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second prophylactic or therapeutic agent to a subject with a disorder.

Chemotherapy may include Abitrexate (Methotrexate Injection), Abraxane (Paclitaxel Injection), Adcetris (Brentuximab Vedotin Injection), Adriamycin (Doxorubicin), Adrucil Injection (5-FU (fluorouracil)), Afinitor (Everolimus), Afinitor Disperz (Everolimus), Alimta (PEMET EXED), Alkeran Injection (Melphalan Injection), Alkeran Tablets (Melphalan), Aredia (Pamidronate), Arimidex (Anastrozole), Aromasin (Exemestane), Arranon (Nelarabine), Arzerra (Ofatumumab Injection), Avastin (Bevacizumab), Bexxar (Tositumomab), BiCNU (Carmustine), Blenoxane (Bleomycin), Bosulif (Bosutinib), Busulfex Injection (Busulfan Injection), Campath (Alemtuzumab), Camptosar (Irinotecan), Caprelsa (Vandetanib), Casodex (Bicalutamide), CeeNU (Lomustine), CeeNU Dose Pack (Lomustine), Cerubidine (Daunorubicin), Clolar (Clofarabine Injection), Cometriq (Cabozantinib), Cosmegen (Dactinomycin), CytosarU (Cytarabine), Cytoxan (Cytoxan), Cytoxan Injection (Cyclophosphamide Injection), Dacogen (Decitabine), DaunoXome (Daunorubicin Lipid Complex Injection), Decadron (Dexamethasone), DepoCyt (Cytarabine Lipid Complex Injection), Dexamethasone Intensol (Dexamethasone), Dexpak Taperpak (Dexamethasone), Docefrez (Docetaxel), Doxil (Doxorubicin Lipid Complex Injection), Droxia (Hydroxyurea), DTIC (Decarbazine), Eligard (Leuprolide), Ellence (Ellence (epirubicin)), Eloxatin (Eloxatin (oxaliplatin)), Elspar (Asparaginase), Emcyt (Estramustine), Erbitux (Cetuximab), Erivedge (Vismodegib), Erwinaze (Asparaginase Erwinia chrysanthemi), Ethyol (Amifostine), Etopophos (Etoposide Injection), Eulexin (Flutamide), Fareston (Toremifene), Faslodex (Fulvestrant), Femara (Letrozole), Firmagon (Degarelix Injection), Fludara (Fludarabine), Folex (Methotrexate Injection), Folotyn (Pralatrexate Injection), FUDR (FUDR (floxuridine)), Gemzar (Gemcitabine), Gilotrif (Afatinib), Gleevec (Imatinib Mesylate), Gliadel Wafer (Carmustine wafer), Halaven (Eribulin Injection), Herceptin (Trastuzumab), Hexalen (Altretamine), Hycamtin (Topotecan), Hycamtin (Topotecan), Hydrea (Hydroxyurea), Iclusig (Ponatinib), Idamycin PFS (Idarubicin), Ifex (Ifosfamide), Inlyta (Axitinib), Intron A alfab (Interferon alfa-2a), Iressa (Gefitinib), Istodax (Romidepsin Injection), Ixempra (Ixabepilone Injection), Jakafi (Ruxolitinib), Jevtana (Cabazitaxel Injection), Kadcyla (Ado-trastuzumab Emtansine), Kyprolis (Carfilzomib), Leukeran (Chlorambucil), Leukine (Sargramostim), Leustatin (Cladribine), Lupron (Leuprolide), Lupron Depot (Leuprolide), Lupron DepotPED (Leuprolide), Lysodren (Mitotane), Marqibo Kit (Vincristine Lipid Complex Injection), Matulane (Procarbazine), Megace (Megestrol), Mekinist (Trametinib), Mesnex (Mesna), Mesnex (Mesna Injection), Metastron (Strontium-89 Chloride), Mexate (Methotrexate Injection), Mustargen (Mechlorethamine), Mutamycin (Mitomycin), Myleran (Busulfan), Mylotarg (Gemtuzumab Ozogamicin), Navelbine (Vinorelbine), Neosar Injection (Cyclophosphamide Injection), Neulasta (filgrastim), Neulasta (pegfilgrastim), Neupogen (filgrastim), Nexavar (Sorafenib), Nilandron (Nilandron (nilutamide)), Nipent (Pentostatin), Nolvadex (Tamoxifen), Novantrone (Mitoxantrone), Oncaspar (Pegaspargase), Oncovin (Vincristine), Ontak (Denileukin Diftitox), Onxol (Paclitaxel Injection), Panretin (Alitretinoin), Paraplatin (Carboplatin), Perjeta (Pertuzumab Injection), Platinol (Cisplatin), Platinol (Cisplatin Injection), PlatinolAQ (Cisplatin), PlatinolAQ (Cisplatin Injection), Pomalyst (Pomalidomide), Prednisone Intensol (Prednisone), Proleukin (Aldesleukin), Purinethol (Mercaptopurine), Reclast (Zoledronic acid), Revlimid (Lenalidomide), Rheumatrex (Methotrexate), Rituxan (Rituximab), RoferonA alfaa (Interferon alfa-2a), Rubex (Doxorubicin), Sandostatin (Octreotide), Sandostatin LAR Depot (Octreotide), Soltamox (Tamoxifen), Sprycel (Dasatinib), Sterapred (Prednisone), Sterapred DS (Prednisone), Stivarga (Regorafenib), Supprelin LA (Histrelin Implant), Sutent (Sunitinib), Sylatron (Peginterferon Alfa-2b Injection (Sylatron)), Synribo (Omacetaxine Injection), Tabloid (Thioguanine), Taflinar (Dabrafenib), Tarceva (Erlotinib), Targretin Capsules (Bexarotene), Tasigna (Decarbazine), Taxol (Paclitaxel Injection), Taxotere (Docetaxel), Temodar (Temozolomide), Temodar (Temozolomide Injection), Tepadina (Thiotepa), Thalomid (Thalidomide), TheraCys BCG (BCG), Thioplex (Thiotepa), TICE BCG (BCG), Toposar (Etoposide Injection), Torisel (Temsirolimus), Treanda (Bendamustine hydrochloride), Trelstar (Triptorelin Injection), Trexall (Methotrexate), Trisenox (Arsenic trioxide), Tykerb (lapatinib), Valstar (Valrubicin Intravesical), Vantas (Histrelin Implant), Vectibix (Panitumumab), Velban (Vinblastine), Velcade (Bortezomib), Vepesid (Etoposide), Vepesid (Etoposide Injection), Vesanoid (Tretinoin), Vidaza (Azacitidine), Vincasar PFS (Vincristine), Vincrex (Vincristine), Votrient (Pazopanib), Vumon (Teniposide), Wellcovorin IV (Leucovorin Injection), Xalkori (Crizotinib), Xeloda (Capecitabine), Xtandi (Enzalutamide), Yervoy (Ipilimumab Injection), Zaltrap (Ziv-aflibercept Injection), Zanosar (Streptozocin), Zelboraf (Vemurafenib), Zevalin (Ibritumomab Tiuxetan), Zoladex (Goserelin), Zolinza (Vorinostat), Zometa (Zoledronic acid), Zortress (Everolimus), Zytiga (Abiraterone), Nimotuzumab and immune checkpoint inhibitors such as nivolumab, pembrolizumab/MK-3475, pidilizumab and AMP-224 targeting PD-1; and BMS-935559, MEDI4736, MPDL3280A and MSB0010718C targeting PD-L1 and those targeting CTLA-4 such as ipilimumab.

Antibiotics, e.g. antibiotics with the classes of aminoglycosides; carbapenems; and the like; penicillins, e.g. penicillin G, penicillin V, methicillin, oxacillin, carbenicillin, nafcillin, ampicillin, etc. penicillins in combination with ⊐-lactamase inhibitors, cephalosporins, e.g. cefaclor, cefazolin, cefuroxime, moxalactam, etc; tetracyclines; cephalosporins; quinolones; lincomycins; macrolides; sulfonamides; glycopeptides including the anti-infective antibiotics vancomycin, teicoplanin, telavancin, ramoplanin and decaplanin. Derivatives of vancomycin include, for example, oritavancin and dalbavancin (both lipoglycopeptides). Telavancin is a semi-synthetic lipoglycopeptide derivative of vancomycin (approved by FDA in 2009). Other vancomycin analogs are disclosed, for example, in WO 2015022335 A1 and Chen et al. (2003) PNAS 100 (10): 5658-5663, each herein specifically incorporated by reference. Non-limiting examples of antibiotics include vancomycin, linezolid, azithromycin, daptomycin, colistin, eperezolid, fusidic acid, rifampicin, tetracyclin, fidaxomicin, clindamycin, lincomycin, rifalazil, and clarithromycin.

Radiotherapy means the use of radiation, usually X-rays, to treat illness. X-rays were discovered in 1895 and since then radiation has been used in medicine for diagnosis and investigation (X-rays) and treatment (radiotherapy). Radiotherapy may be from outside the body as external radiotherapy, using X-rays, cobalt irradiation, electrons, and more rarely other particles such as protons. It may also be from within the body as internal radiotherapy, which uses radioactive metals or liquids (isotopes) to treat cancer.

As used herein, endpoints for treatment will be given a meaning as known in the art and as used by the Food and Drug Administration.

Overall survival is defined as the time from randomization until death from any cause, and is measured in the intent-to-treat population. Survival is considered the most reliable cancer endpoint, and when studies can be conducted to adequately assess survival, it is usually the preferred endpoint. This endpoint is precise and easy to measure, documented by the date of death. Bias is not a factor in endpoint measurement. Survival improvement should be analyzed as a risk-benefit analysis to assess clinical benefit. Overall survival can be evaluated in randomized controlled studies. Demonstration of a statistically significant improvement in overall survival can be considered to be clinically significant if the toxicity profile is acceptable, and has often supported new drug approval. A benefit of the methods of the invention can include increased overall survival of patients.

Endpoints that are based on tumor assessments include DFS, ORR, TTP, PFS, and time-to-treatment failure (TTF). The collection and analysis of data on these time-dependent endpoints are based on indirect assessments, calculations, and estimates (e.g., tumor measurements). Disease-Free Survival (DFS) is defined as the time from randomization until recurrence of tumor or death from any cause. The most frequent use of this endpoint is in the adjuvant setting after definitive surgery or radiotherapy. DFS also can be an important endpoint when a large percentage of patients achieve complete responses with chemotherapy.

Objective Response Rate. ORR is defined as the proportion of patients with tumor size reduction of a predefined amount and for a minimum time period. Response duration usually is measured from the time of initial response until documented tumor progression. Generally, the FDA has defined ORR as the sum of partial responses plus complete responses. When defined in this manner, ORR is a direct measure of drug antitumor activity, which can be evaluated in a single-arm study.

Time to Progression and Progression-Free Survival. TTP and PFS have served as primary endpoints for drug approval. TTP is defined as the time from randomization until objective tumor progression; TTP does not include deaths. PFS is defined as the time from randomization until objective tumor progression or death. The precise definition of tumor progression is important and should be carefully detailed in the protocol.

As used herein, the term “correlates,” or “correlates with,” and like terms, refers to a statistical association between instances of two events, where events include numbers, data sets, and the like. For example, when the events involve numbers, a positive correlation (also referred to herein as a “direct correlation”) means that as one increases, the other increases as well. A negative correlation (also referred to herein as an “inverse correlation”) means that as one increases, the other decreases.

“Dosage unit” refers to physically discrete units suited as unitary dosages for the particular individual to be treated. Each unit can contain a predetermined quantity of active compound(s) calculated to produce the desired therapeutic effect(s) in association with the required pharmaceutical carrier. The specification for the dosage unit forms can be dictated by (a) the unique characteristics of the active compound(s) and the particular therapeutic effect(s) to be achieved, and (b) the limitations inherent in the art of compounding such active compound(s).

“Pharmaceutically acceptable excipient” means an excipient that is useful in preparing a pharmaceutical composition that is generally safe, non-toxic, and desirable, and includes excipients that are acceptable for veterinary use as well as for human pharmaceutical use. Such excipients can be solid, liquid, semisolid, or, in the case of an aerosol composition, gaseous.

“Pharmaceutically acceptable salts and esters” means salts and esters that are pharmaceutically acceptable and have the desired pharmacological properties. Such salts include salts that can be formed where acidic protons present in the compounds are capable of reacting with inorganic or organic bases. Suitable inorganic salts include those formed with the alkali metals, e.g. sodium and potassium, magnesium, calcium, and aluminum. Suitable organic salts include those formed with organic bases such as the amine bases, e.g., ethanolamine, diethanolamine, triethanolamine, tromethamine, N methylglucamine, and the like. Such salts also include acid addition salts formed with inorganic acids (e.g., hydrochloric and hydrobromic acids) and organic acids (e.g., acetic acid, citric acid, maleic acid, and the alkane- and arene-sulfonic acids such as methanesulfonic acid and benzenesulfonic acid). Pharmaceutically acceptable esters include esters formed from carboxy, sulfonyloxy, and phosphonoxy groups present in the compounds, e.g., C1-6 alkyl esters. When there are two acidic groups present, a pharmaceutically acceptable salt or ester can be a mono-acid-mono-salt or ester or a di-salt or ester; and similarly where there are more than two acidic groups present, some or all of such groups can be salified or esterified. Compounds named in this invention can be present in unsalified or unesterified form, or in salified and/or esterified form, and the naming of such compounds is intended to include both the original (unsalified and unesterified) compound and its pharmaceutically acceptable salts and esters. Also, certain compounds named in this invention may be present in more than one stereoisomeric form, and the naming of such compounds is intended to include all single stereoisomers and all mixtures (whether racemic or otherwise) of such stereoisomers.

The terms “pharmaceutically acceptable”, “physiologically tolerable” and grammatical variations thereof, as they refer to compositions, carriers, diluents and reagents, are used interchangeably and represent that the materials are capable of administration to or upon a human without the production of undesirable physiological effects to a degree that would prohibit administration of the composition.

A “therapeutically effective amount” means the amount that, when administered to a subject for treating a disease, is sufficient to effect treatment for that disease.

### METHODS OF USE

Methods are provided for treating or reducing primary or metastatic cancer, e.g. a solid tumor, in a regimen comprising contacting the targeted cancer with a combination of (i) an agent that blockades CD47 activity; and (ii) an inhibitor of CXCR2, including without limitation small molecule orally administered inhibitors. Such methods include administering to a subject in need of treatment a therapeutically effective amount or an effective dose of the combined agents of the invention. The combination may further include treatment with a chemotherapeutic drug, radiation therapy, or an ESA. The combination may provide for a synergistic effect, e.g. in the treatment of solid tumors.

Effective doses of the combined agents of the present invention for the treatment of cancer vary depending upon many different factors, including means of administration, target site, physiological state of the patient, whether the patient is human or an animal, other medications administered, and whether treatment is prophylactic or therapeutic. Usually, the patient is a human, but nonhuman mammals may also be treated, e.g. companion animals such as dogs, cats, horses, etc., laboratory mammals such as rabbits, mice, rats, etc., and the like. Treatment dosages can be titrated to optimize safety and efficacy.

In some embodiments, the therapeutic dosage of each agent may range from about 0.0001 to 100 mg/kg, and more usually 0.01 to 5 mg/kg, of the host body weight. For example dosages can be 1 mg/kg body weight or 10 mg/kg body weight or within the range of 1-10 mg/kg. An exemplary treatment regime entails administration once every two weeks or once a month or once every 3 to 6 months. Therapeutic entities of the present invention are usually administered on multiple occasions. Intervals between single dosages can be weekly, monthly or yearly. Intervals can also be irregular as indicated by measuring blood levels of the therapeutic entity in the patient. Alternatively, therapeutic entities of the present invention can be administered as a sustained release formulation, in which case less frequent administration is required. Dosage and frequency vary depending on the half-life of the polypeptide in the patient.

Methods of the present invention include treating, reducing or preventing tumor growth, tumor metastasis or tumor invasion of cancers including carcinomas, lymphomas, melanomas, sarcomas, gliomas, etc. For prophylactic applications, pharmaceutical compositions or medicaments are administered to a patient susceptible to, or otherwise at risk of disease in an amount sufficient to eliminate or reduce the risk, lessen the severity, or delay the outset of the disease, including biochemical, histologic and/or behavioral symptoms of the disease, its complications and intermediate pathological phenotypes presenting during development of the disease.

Compositions for the treatment of cancer can be administered by oral, parenteral, topical, intravenous, intratumoral, subcutaneous, intraarterial, intracranial, intraperitoneal, intranasal or intramuscular means. A typical route of administration is intravenous or intratumoral, although other routes can be equally effective.

Typically, antibody compositions are prepared as injectables, either as liquid solutions or suspensions; solid forms suitable for solution in, or suspension in, liquid vehicles prior to injection can also be prepared. The preparation also can be emulsified or encapsulated in liposomes or micro particles such as polylactide, polyglycolide, or copolymer for enhanced adjuvant effect, as discussed above. Langer, Science 249: 1527, 1990 and Hanes, Advanced Drug Delivery Reviews 28: 97-119, 1997. The agents of this invention can be administered in the form of a depot injection or implant preparation which can be formulated in such a manner as to permit a sustained or pulsatile release of the active ingredient. The pharmaceutical compositions are generally formulated as sterile, substantially isotonic and in full compliance with all Good Manufacturing Practice (GMP) regulations of the U.S. Food and Drug Administration.

Toxicity of the combined agents described herein can be determined by standard pharmaceutical procedures in cell cultures or experimental animals, e.g., by determining the LD₅₀ (the dose lethal to 50% of the population) or the LD₁₀₀ (the dose lethal to 100% of the population). The dose ratio between toxic and therapeutic effect is the therapeutic index. The data obtained from these cell culture assays and animal studies can be used in formulating a dosage range that is not toxic for use in human. The dosage of the proteins described herein lies preferably within a range of circulating concentrations that include the effective dose with little or no toxicity. The dosage can vary within this range depending upon the dosage form employed and the route of administration utilized. The exact formulation, route of administration and dosage can be chosen by the individual physician in view of the patient's condition.

The pharmaceutical compositions can be administered in a variety of unit dosage forms depending upon the method of administration. For example, unit dosage forms suitable for oral administration include, but are not limited to, powder, tablets, pills, capsules and lozenges. It is recognized that compositions of the invention when administered orally, should be protected from digestion. This is typically accomplished either by complexing the molecules with a composition to render them resistant to acidic and enzymatic hydrolysis, or by packaging the molecules in an appropriately resistant carrier, such as a liposome or a protection barrier. Means of protecting agents from digestion are well known in the art.

The compositions for administration will commonly comprise an antibody or an inhibitor of CXCR2 dissolved in a pharmaceutically acceptable carrier, preferably an aqueous carrier. A variety of aqueous carriers can be used, e.g., buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents and the like, e.g., sodium acetate, sodium chloride, potassium chloride, calcium chloride, sodium lactate and the like. The concentration of active agent in these formulations can vary widely, and will be selected primarily based on fluid volumes, viscosities, body weight and the like in accordance with the particular mode of administration selected and the patient's needs (e.g., Remington's Pharmaceutical Science (15th ed., 1980) and Goodman & Gillman, The Pharmacological Basis of Therapeutics (Hardman et al., eds., 1996)).

Also within the scope of the invention are kits comprising the active agents and formulations thereof, of the invention and instructions for use. The kit can further contain a least one additional reagent, e.g. a chemotherapeutic drug, ESA, etc. Kits typically include a label indicating the intended use of the contents of the kit. The term label includes any writing, or recorded material supplied on or with the kit, or which otherwise accompanies the kit.

The compositions can be administered for therapeutic treatment. Compositions are administered to a patient in an amount sufficient to substantially ablate targeted cells, as described above. An amount adequate to accomplish this is defined as a “therapeutically effective dose”, which may provide for an improvement in overall survival rates. Single or multiple administrations of the compositions may be administered depending on the dosage and frequency as required and tolerated by the patient. The particular dose required for a treatment will depend upon the medical condition and history of the mammal, as well as other factors such as age, weight, gender, administration route, efficiency, etc.

### Experimental

Colony stimulating factor-1 receptor (CSF1R) signaling is crucial for macrophage differentiation and survival. Inhibition of CSF1R using monoclonal antibodies and antagonists has been widely explored as a cancer immunotherapy due to its robust depletion of pro-tumorigenic tumor-associated macrophages (TAMs). However, therapeutic blockade of CSF1R demonstrates limited clinical efficacy, and the effects of CSF1R inhibition on myelopoiesis require deeper investigation. In this study, we report that deletion of CSF1R in an inducible knockout model leads to robust expansion of CXCR2+ granulocytic-myeloid derived suppressor cells (G-MDSCs). in thioglycolate-induced peritonitis and tumorigenesis. These data show that the therapeutic benefits of CSF1R blockade are dampened by elevated G-MDSC tumor infiltration. Further, this study demonstrates that targeting CXCR2+ G-MDSCs in combination with anti-CD47 monoclonal antibody treatment provides an effective therapeutic strategy for treatment of solid tumors.

To determine the effects of CSF1R deletion in myelopoiesis and tumor progression, we developed and used an inducible CSF1R knockout model. Although robust reduction of macrophages is achieved, this data shows that deletion of CSF1R can lead to increased expansion of CXCR2+ G-MDSCs during inflammation and tumorigenesis, resulting in accelerated tumor progression. Further, our data also demonstrates that using a small molecule CXCR2 antagonist can promote an anti-tumor response which is further enhanced by CD47 blockade.

Generation and validation of inducible CSF1R knockout mice. Therapeutic approaches to inhibit CSF1R or its ligands include the use of monoclonal antibodies and small molecule antagonists. To study the role of CSF1R and the consequences of its deletion, we sought to use a genetic knockout system as an alternative approach to pharmacological agents. Deletion of the CSF1R locus in the mouse and rat germline results in a significant reduction of tissue resident macrophages. However, CSF1R^{-/-}mice suffer significantly impaired postnatal growth, become osteoporotic, and rarely survive adulthood.

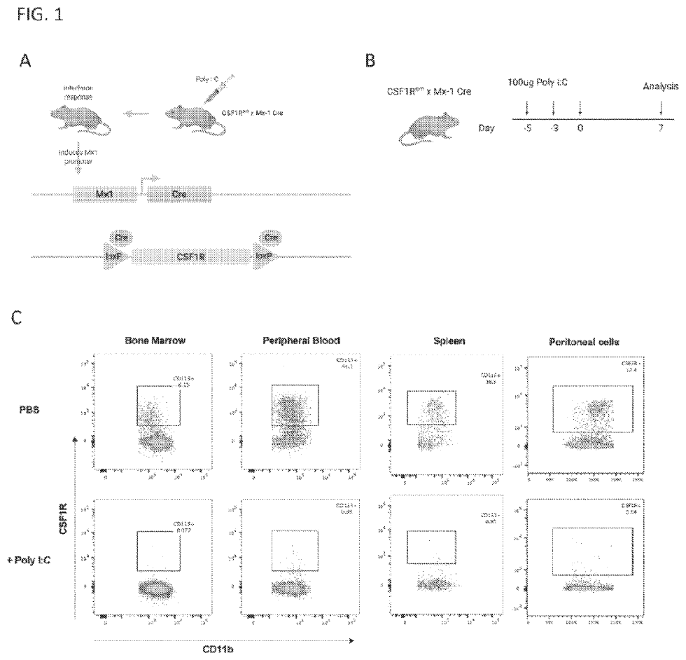

To study loss of CSF1R in adult mice, we developed an inducible CSF1R knockout mouse. To achieve inducible deletion, CSF1R^{FI/FI} mice were crossed with Mx1-Cre mice. In this system, the Mx1 promoter is activated in an interferon-dependent manner following intraperitoneal injection of Polyinosinic: polycytidylic acid (Poly I:C), a TLR3 agonist (FIG. 1A). Mice received intraperitoneal injections of Poly I:C every other day for a total of three injections. To validate deletion of CSF1R, CSF1R^{FI/FI}-Mx1-Cre mice were treated with Poly I:C and were allowed to rest for one week to allow their interferon response to subside (FIG. 1B). Peripheral blood, bone marrow, spleen, and peritoneal cells were then harvested and analyzed by flow cytometry. Loss of CSF1R was observed on CD11b+ cells, confirming its deletion following administration of Poly I:C (FIG. 1C).

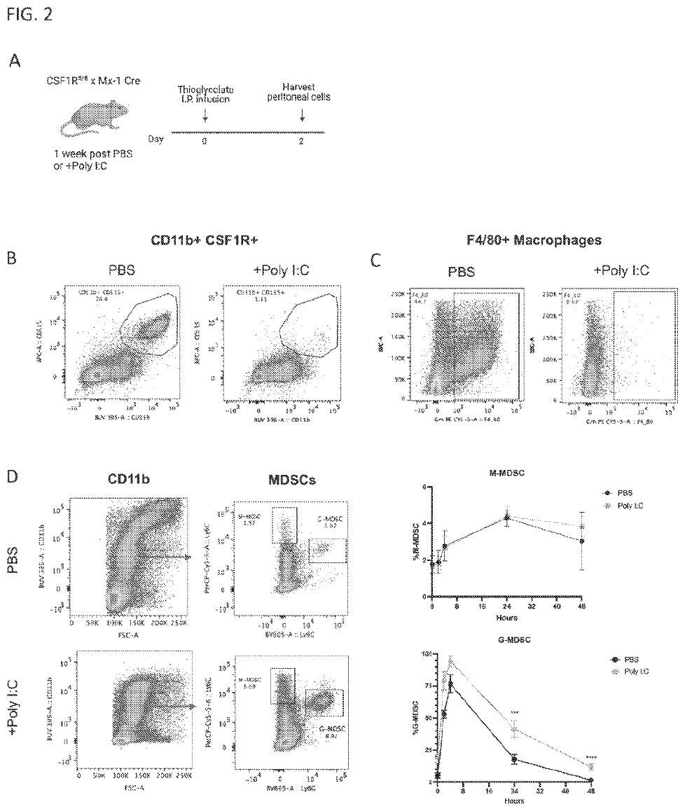

Deletion of CSF1R leads to increased G-MDSCs in thioglycolate-induced peritonitis. Under steady state conditions, the mouse peritoneal cavity contains a steady population of tissue resident macrophages. In models of sterile inflammation, such as the induction of peritonitis by administration of thioglycolate, monocytes from the periphery are rapidly recruited to the peritoneum where they differentiate into macrophages. To determine whether monocyte-derived macrophages could be generated and recruited to the peritoneum in this system, Poly I:C-treated CSF1R^{FI/FI}-Mx1-Cre mice were infused with thioglycolate. 48 hours after infusion, peritoneal cells were harvested and subjected to flow cytometry analysis (FIG. 2A). CSF1R expression was not found on CD11b+ cells, and a significant reduction in the F4/80+ macrophage population was demonstrated in comparison to the control group (FIG. 2B). Within the CD11b population, significantly increased levels of G-MDSCs were observed in Poly I:C-treated mice. No significant differences were observed in M-MDSCs (CD11b+Ly6C+Ly6G−). When observed over a time course of 48 hrs, G-MDSCs were consistently significantly increased in Poly I:C-treated mice (FIG. 2C). No significant differences in M-MDSCs were observed between control and Poly I:C-treated mice.

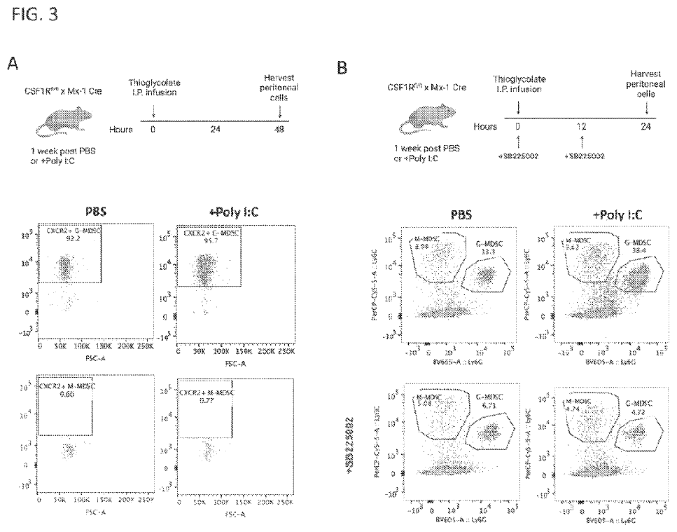

Blocking G-MDSC recruitment in thioglycolate-induced peritonitis with a CXCR2 inhibitor. The C-X-C chemokine receptor 2 (CXCR2) signaling axis plays a key role in the migration of immunosuppressive MDSCs. To identify whether these subsets of MDSCs in the peritoneal cavity elicited through thioglycolate-induced peritonitis express CXCR2, peritoneal cells were harvested 24 hours after intraperitoneal injection of thioglycolate and were analyzed by flow cytometry (FIG. 3A). G-MDSCs highly expressed CXCR2, while M-MDSCs did not. We next sought to determine whether CXCR2+ G-MDSCs could be targeted to block their recruitment to the peritoneum in response to thioglycolate. Control and Poly I:C-treated mice were injected ip with thioglycolate and with or without SB225002, a CXCR2 inhibitor. 12 hr later, mice were treated again with or without SB225002. At the 24 h time point, peritoneal cells were harvested and analyzed by flow cytometry (FIG. 3B). Mice that received IP treatments of SB255002 demonstrated a significantly decreased percentage of G-MDSCs in the peritoneal cavity.

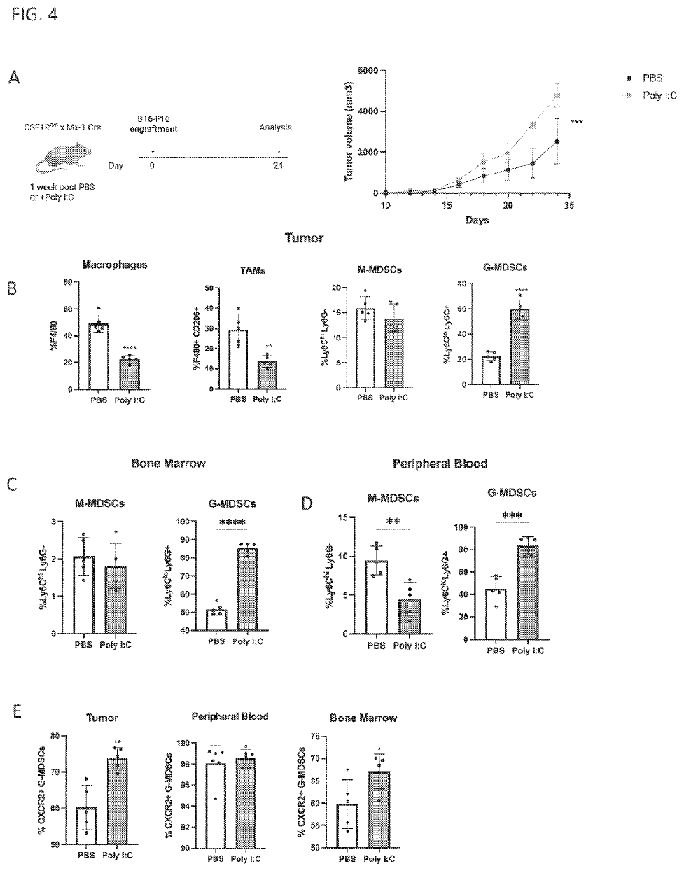

Loss of CSF1R leads to increased G-MDSCs in a model of B16-F10 melanoma. To identify whether increased G-MDSC expansion and infiltration could also be observed in tumors of CSF1R deficient mice, we subcutaneously engrafted B16-F10 melanoma into PBS or poly I:C-treated CSF1R^{FI/FI}-Mx1-Cre mice. Mice lacking CSF1R had significantly larger tumors than the PBS-treated group (FIG. 4A). To determine how loss of CSF1R expression resulted in increased tumor progression, tumors were subjected to immunophenotyping by flow cytometry. Mice lacking CSF1R showed a significant reduction in their F4/80+ tumor associated macrophage population (FIG. 4B). While no significant difference was observed in the M-MDSC (CD11b+Ly6C+Ly6G−) populations, there was a significant increase in the G-MDSC (CD11b+Ly6C^{lo} Ly6G+) population in mice lacking CSF1R (FIG. 4C). Further, tumor-infiltrating G-MDSCs were found to be CXCR2+ (FIG. 4D). Bone marrow, spleen, and peripheral blood from CSF1R-deficient tumor-bearing mice also showed significantly increased CXCR2+ G-MDSCs compared to control (FIG. 4E).

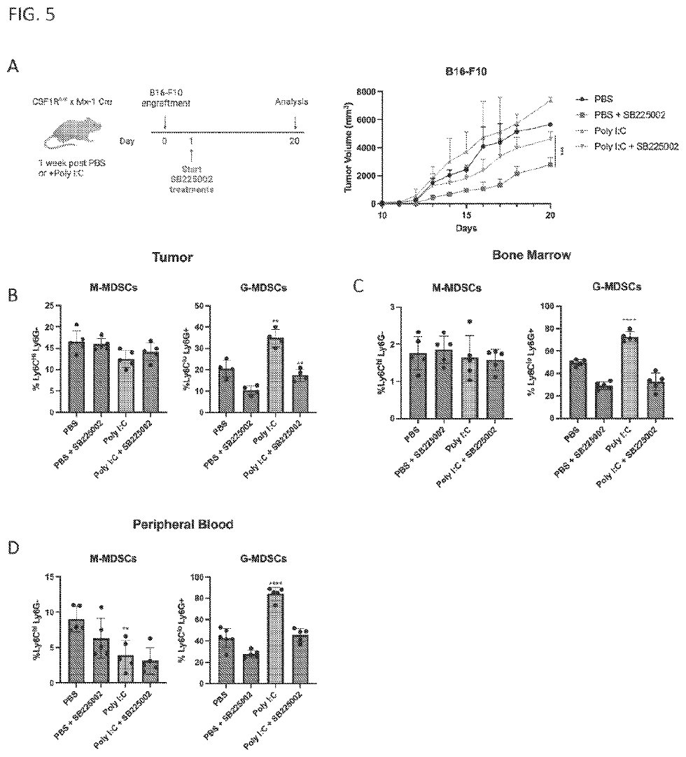

Targeting G-MDSCs in B16-F10 melanoma with a CXCR2 inhibitor. To determine whether targeting CXCR2+ G-MDSCs could lead to an anti-tumor response, poly I:C and PBS mice were subcutaneously engrafted with B16-F10 melanoma. The day after engraftment, mice were treated intraperitoneally with PBS or SB225002 six days per week. Treatment with SB225002 significantly decreased tumor progression, especially in mice expressing CSF1R (FIG. 5A). Tumors were harvested and subjected to flow cytometry for immunophenotyping. A significant decrease in tumor-infiltrating G-MDSCs was observed in mice that were treated with SB225002 (FIG. 5B). Further, a significant decrease of G-MDSCs was found in the bone marrow and peripheral blood of tumor-bearing mice (FIG. 5C).

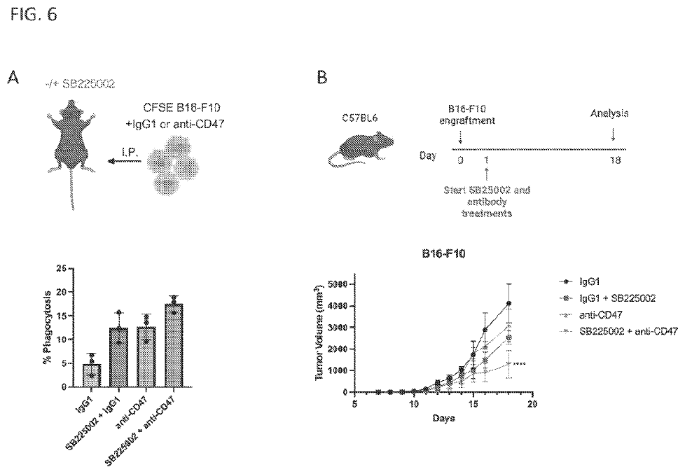

Targeting G-MDSCs in combination with CD47 blockade enhances anti-tumor response. Previous studies have demonstrated that MDSCs are capable of dampening inflammatory responses by the immune system through various mechanisms. We sought to determine whether CXCR2+ G-MDSCs are capable of suppressing macrophage phagocytosis in vivo. Wild-type C57BL6 mice were injected with thioglycolate and either received or did not receive two treatments of SB225002 (0 hrs, 12 hrs) to deplete G-MDSCs in the peritoneal cavity. B16-F10 melanoma cells were either pre-treated with control mouse IgG1 or anti-CD47 mAb MIAP410 and were injected ip into the mice. Peritoneal cells were recovered after 4 hours and phagocytosis by F4/80+ macrophages was measured by flow cytometry (FIG. 6A). Macrophages from mice that received SB225002 treatments and anti-CD47 pre-opsonized B16-F10 cells demonstrated the highest phagocytic capacity.

To determine whether the combination of SB225002 and anti-CD47 could lead to an enhanced anti-tumor response, wild-type C57BI/6 mice were subcutaneously engrafted with B16-F10 melanoma. Mice that received combination treatment had significantly smaller tumors than SB225002 or anti-CD47 alone (FIG. 6B).

The tumor microenvironment contains a variety of immunosuppressive cells that assist in tumorigenesis and dampen immune response. One such cell type, TAMs, have gained notable attention due to their abundance in tumors, plasticity, and immunosuppressive functions. Significant efforts have focused on the development of CSF1R inhibitors in an attempt to relieve the tumor microenvironment from immunosuppression. However, CSF1R inhibition has shown limited therapeutic benefits and despite our growing understanding of TAM contribution to tumor progression, the consequences of their depletion have been unclear.

The data presented here demonstrates that deletion of CSF1R leads to increased expansion and recruitment of CXCR2+ G-MDSCs in both models of peritonitis and B16-F10 melanoma. Thus, we have uncovered a pathway by which tumor growth may persist in the presence of CSF1R deletion: through the influx of G-MDSCs in both the primary tumor site, as well as throughout circulation.

In healthy individuals, immature myeloid cells that are generated in the bone marrow differentiate into mature granulocytes, macrophages, or dendritic cells. However, in pathological conditions such as infectious disease and cancer, these immature myeloid cells rapidly expand into a heterogenous population that has potent immunosuppressive activity, known as MDSCs. The heterogeneity of MDSCs of can be divided into two main subsets: G-MDSCs, which have a CD11b⁺Ly6G⁺Ly6C^{low} phenotype and M-MDSCs with monocytic morphology are CD11b⁺Ly6G⁻Ly6C^{hi}.

Greater accumulation of MDSCs within the periphery or tumors of patients has been shown to correlate with tumor progression and poor prognosis in multiple malignancies. The CXCR2/CXCL1 signaling axis has recently been shown to play an important role in the recruitment of G-MDSCs. Therefore, we next inhibited CXCR2 in combination with CSF1R deletion and observed decreased G-MDSC recruitment and slower tumor growth. However, CXCR2 inhibition in C57BL6 tumor-bearing mice proved to be more effective in slowing tumor growth, suggesting its promise as a single agent therapeutic. Moreover, this data also suggests that TAMs may be an important component involved in restricting further infiltration of G-MDSCs.

We also found that targeting CXCR2+ G-MDSCs in an in vivo phagocytosis assay led to increased phagocytosis of B16-F10 melanoma cells by macrophages, which was further enhanced by anti-CD47 opsonization. C57BL6 mice subcutaneously engrafted with B16-F10 that were treated with a combination of CXCR2 inhibitor and anti-CD47 demonstrated robust anti-tumor responses. This further suggests that G-MDSCs may dampen macrophage anti-tumor activity and targeting them may lead to delayed tumor progression through increased tumor clearance by macrophages.

In summary, we demonstrate that G-MDSCs are the dominant immunosuppressive myeloid cell subtype within B16-F10 melanoma tumors. Depletion of TAMs may not completely resolve immunosuppression, as G-MDSCs expand as a result. Targeting CXCR2+ G-MDSCs in combination with CD47 blockade may be an ideal approach for selectively abrogating G-MDSC trafficking into tumors while restoring macrophage phagocytosis of tumor cells.

### Methods

Mice. C57BI6/J mice, B6.Cg-Csf1r^{tm1.2Jwp}/J (Csf1r^{fl/f}) mice, and B6.Cg-Tg (Mx1-cre)1Cgn/J mice were obtained from The Jackson Laboratory. Mice were bred and maintained at the Stanford University Research Animal Facility. All experiments were carried out in accordance with ethical care guidelines set by the Stanford University Administrative Panel on Laboratory Animal Care (APLAC).

Cell culture and reagents. B16-F10 murine melanoma cells were purchased from the American Type Culture Collection (ATCC) and were cultured in DMEM+GlutaMax+10% fetal bovine serum+100U ml penicillin/streptomycin. All cells were cultured in a humidified, 5% CO₂ incubator at 37° C. SB225002 was purchased from Tocris (Cat. No. 2725) and Poly I:C HMW was obtained from Invivogen (tlrl-pic).

Flow cytometry. Samples were blocked with monoclonal antibody to CD16/32 (TruStain fcX, BioLegend) before staining with antibody panels. Samples were stained for 30 min on ice, and subsequently washed with FACS buffer (PBS with 1% FBS buffer). Fluorescence compensations were performed using single-stained UltraComp eBeads (Affymetrix). The following antibodies were used for FACS analysis: Anti-CD11b (M1/70 BD Biosciences); Anti-F4/80 (BM8 BioLegend); Anti-Ly6G (1A8 BioLegend), Anti-Ly6C (HK1.4 BioLegend); Anti-CXCR2 (SA044G4 BioLegend). SYTOX blue dead cell stain (Invitrogen) was used for dead cell exclusion. Data was acquired using a FACSAria II cell sorter (BD Biosciences) and analyzed using FlowJo software.

Thioglycolate induced peritonitis and isolation of peritoneal cells. Peritoneal lavage was conducted at indicated times after intraperitoneal injection of 1 mL of 3% thioglycolate medium (Difco). Cells were resuspended in FACS buffer and were stained with antibodies as previously described for flow cytometry analysis.

In vivo phagocytosis assay. B16-F10 melanoma cells were labeled with CellTrace CFSE (Thermo Fisher Scientific) according to manufacturer's recommendations. CFSE labeled B16-F10 cells were incubated with IgG1 isotype control or anti-CD47 (MIAP410) for 20 min on ice and washed with PBS. 1×10⁶ labeled cells were subsequently injected into the peritoneal cavity of C57BL6 mice. Four hours after the injections, mice were euthanized, and cells were harvested by peritoneal lavage. Peritoneal macrophages were stained with anti-F4/80 for 30 min on ice and washed with FACS buffer. Phagocytosis was assessed by flow cytometry.

In vivo tumor experiments and treatments. Mice 6-8 weeks of age were given subcutaneous injection of B16-F10 melanoma cells in PBS into the right flank. Tumor growth was measured by using calipers and volumes were calculated by using the formula: volume= 4/3π×(x/2)2×(y/2), where x is the largest measurable dimension of the tumor and y is the dimension immediately perpendicular to x. Tumor volumes were evaluated using two-way ANOVA in GraphPad Prism. For tumor treatments, mice were given mouse IgG1 isotype control clone MOPC21 (BioXCell), anti-CD47 mAb clone MIAP410 (BioXCell), or SB225002 (ToCris) by intraperitoneal injection.

Statistical analysis and graphing software. All data collected were statistically analyzed and graphed using GraphPad Prism software.

### REFERENCES

Yang Y. Cancer immunotherapy: harnessing the immune system to battle cancer. The Journal of Clinical Investigation. 2015; 125(9):3335-3337. doi: 10.1172/JCI83871.
Sharma, P., Hu-Lieskovan, S., Wargo, J. A., & Ribas, A. (2017). Primary, Adaptive, and Acquired Resistance to Cancer Immunotherapy. Cell, 168(4), 707-723.
Waldman, A.D., Fritz, J.M. & Lenardo, M.J. A guide to cancer immunotherapy: from T cell basic science to clinical practice. Nat Rev Immunol 20, 651-668 (2020). https://doi.org/10.1038/s41577-020-0306-5
Demaria, O., Cornen, S., Daëron, M. et al. Harnessing innate immunity in cancer therapy. Nature 574, 45-56 (2019).
Rameshbabu, S., Labadie, B. W., Argulian, A., & Patnaik, A. (2021). Targeting InnateImmunity in Cancer Therapy. Vaccines, 9(2), 138.
Jaiswal, S., Chao, M. P., Majeti, R., & Weissman, I. L. (2010). Macrophages as mediators of tumor immunosurveillance. Trends in immunology, 31(6), 212-219. 7. Chao, M., Majeti, R. & Weissman, I. Programmed cell removal: a new obstacle in the road to developing cancer. Nat Rev Cancer 12, 58-67 (2012). https://doi.org/10.1038/nrc3171
Feng, M., Marjon, K.D., Zhu, F. et al. Programmed cell removal by calreticulin in tissue homeostasis and cancer. Nat Commun 9, 3194 (2018).
Willingham, et al. (2012). The CD47-signal regulatory protein alpha (SIRPa) interaction is a therapeutic target for human solid tumors. Proceedings of the National Academy of Sciences of the United States of America, 109(17), 6662-6667.
Jaiswal, S., Jamieson, C. H., Pang, W. W., Park, C. Y., Chao, M. P., Majeti, R., Traver, D., van Rooijen, N., & Weissman, I. L. (2009). CD47 is upregulated on circulating hematopoietic stem cells and leukemia cells to avoid phagocytosis. Cell, 138(2), 271-285.
Pan, Y., Yu, Y., Wang, X., & Zhang, T. (2020). Tumor-Associated Macrophages in Tumor Immunity. Frontiers in immunology, 11, 583084.
Yang, Y., Li, C., Liu, T., Dai, X., & Bazhin, A. V. (2020). Myeloid-Derived Suppressor Cells in Tumors: From Mechanisms to Antigen Specificity and Microenvironmental Regulation. Frontiers in immunology, 11, 1371.
Davidov, V., Jensen, G., Mai, S., Chen, S. H., & Pan, P. Y. (2020). Analyzing One Cell at a TIME: Analysis of Myeloid Cell Contributions in the Tumor Immune Microenvironment. Frontiers in immunology, 11, 1842.
Xiang, X., Wang, J., Lu, D., & Xu, X. (2021). Targeting tumor-associated macrophages to synergize tumor immunotherapy. Signal transduction and targeted therapy, 6(1), 75.
Stanley, E. R., & Chitu, V. (2014). CSF-1 receptor signaling in myeloid cells. Cold Spring Harbor perspectives in biology, 6(6), a021857.
Muñoz-Garcia, J., Cochonneau, D., Télétchéa, S., Moranton, E., Lanoe, D., Brion, R., Lézot, F., Heymann, M. F., & Heymann, D. (2021). The twin cytokines interleukin-34 and CSF-1: masterful conductors of macrophage homeostasis. Theranostics, 11(4), 1568-1593.
Cannarile, M. A., Weisser, M., Jacob, W., Jegg, A. M., Ries, C. H., & Rüttinger, D. (2017). Colony-stimulating factor 1 receptor (CSF1R) inhibitors in cancer therapy. Journal for immunotherapy of cancer, 5(1), 53.
O'Brien, S. A., Orf, J., Skrzypczynska, K. M., Tan, H., Kim, J., DeVoss, J., Belmontes, B., & Egen, J. G. (2021). Activity of tumor-associated macrophage depletion by CSF1R blockade is highly dependent on the tumor model and timing of treatment. Cancer immunology, immunotherapy: CII, 70(8), 2401-2410.
Papadopoulos, K. P., Gluck, L., Martin, L. P., Olszanski, A. J., Tolcher, A. W., Ngarmchamnanrith, G., Rasmussen, E., Amore, B. M., Nagorsen, D., Hill, J. S., & Stephenson, J., Jr (2017). First-in-Human Study of AMG 820, a Monoclonal Anti-Colony-Stimulating Factor Receptor Antibody, in Patients with Advanced Solid Tumors. Clinical cancer research: an official journal of the American Association for Cancer Research, 23(19), 5703-5710.
Gomez-Roca et al. (2019). Phase I study of emactuzumab single agent or in combination with paclitaxel in patients with advanced/metastatic solid tumors reveals depletion of immunosuppressive M2-like macrophages. Annals of oncology: official journal of the European Society for Medical Oncology, 30(8), 1381-1392.
Dai, X. M., Ryan, G. R., Hapel, A. J., Dominguez, M. G., Russell, R. G., Kapp, S., Sylvestre, V., & Stanley, E. R. (2002). Targeted disruption of the mouse colony-stimulating factor 1 receptor gene results in osteopetrosis, mononuclear phagocyte deficiency, increased primitive progenitor cell frequencies, and reproductive defects. Blood, 99 (1), 111-120.
Pridans C, Raper A, Davis GM, et al. Correction: Pleiotropic Impacts of Macrophage and Microglial Deficiency on Development in Rats with Targeted Mutation of the Csf1r Locus. J Immunol. 2019; 202(11):3334-3335. doi: 10.4049/jimmunol.1900420
Cassado, A., D'Império Lima, M. R., & Bortoluci, K. R. (2015). Revisiting mouse peritoneal macrophages: heterogeneity, development, and function. Frontiers in immunology, 6, 225.
Li, Y. M., Baviello, G., Vlassara, H., & Mitsuhashi, T. (1997). Glycation products in aged thioglycollate medium enhance the elicitation of peritoneal macrophages. Journal of immunological methods, 201(2), 183-188.
Leijh, P. C., van Zwet, T. L., ter Kuile, M. N., & van Furth, R. (1984). Effect of thioglycolate on phagocytic and microbicidal activities of peritoneal macrophages. Infection and immunity, 46(2), 448-452.
Bullock, K., & Richmond, A. (2021). Suppressing MDSC Recruitment to the Tumor Microenvironment by Antagonizing CXCR2 to Enhance the Efficacy of Immunotherapy. Cancers, 13(24), 6293.
Yaseen, M. M., Abuharfeil, N. M., Darmani, H., & Daoud, A. (2020). Mechanisms of immune suppression by myeloid-derived suppressor cells: the role of interleukin-10 as a key immunoregulatory cytokine. Open biology, 10(9), 200111.
Groth, C., Hu, X., Weber, R. et al. Immunosuppression mediated by myeloid-derived suppressor cells (MDSCs) during tumour progression. Br J Cancer 120, 16-25 (2019).
Tang, T., Huang, X., Zhang, G. et al. Advantages of targeting the tumor immune microenvironment over blocking immune checkpoint in cancer immunotherapy. Sig Transduct Target Ther 6, 72 (2021).
Labani-Motlagh, A., Ashja-Mahdavi, M., & Loskog, A. (2020). The Tumor Microenvironment: A Milieu Hindering and Obstructing Antitumor Immune Responses. Frontiers in immunology, 11, 940.
Liu, Y., & Wang, R. (2020). Immunotherapy Targeting Tumor-Associated Macrophages. Frontiers in medicine, 7, 583708.
Petty, A. J., Owen, D. H., Yang, Y., & Huang, X. (2021). Targeting Tumor-Associated Macrophages in Cancer Immunotherapy. Cancers, 13(21), 5318.
Millrud, C. R., Bergenfelz, C., & Leandersson, K. (2017). On the origin of myeloid-derived suppressor cells. Oncotarget, 8(2), 3649-3665.
Gabrilovich, D., Nagaraj, S. Myeloid-derived suppressor cells as regulators of the immune system. Nat Rev Immunol 9, 162-174 (2009).
Bronte, V., Brandau, S., Chen, SH. et al. Recommendations for myeloid-derived suppressor cell nomenclature and characterization standards. Nat Commun 7, 12150 (2016).
Gonda, K., Shibata, M., Ohtake, T., Matsumoto, Y., Tachibana, K., Abe, N., Ohto, H., Sakurai, K., & Takenoshita, S. (2017). Myeloid-derived suppressor cells are increased and correlated with type 2 immune responses, malnutrition, inflammation, and poor prognosis in patients with breast cancer. Oncology letters, 14(2), 1766-1774.
Yang, G., Shen, W., Zhang, Y., Liu, M., Zhang, L., Liu, Q., Lu, H. H., & Bo, J. (2017). Accumulation of myeloid-derived suppressor cells (MDSCs) induced by low levels of IL-6 correlates with poor prognosis in bladder cancer. Oncotarget, 8(24), 38378-38388.
Ai, L., Mu, S., Wang, Y., Wang, H., Cai, L., Li, W., & Hu, Y. (2018). Prognostic role of myeloid-derived suppressor cells in cancers: a systematic review and meta-analysis. BMC cancer, 18(1), 1220.
Highfill, S. L., Cui, Y., Giles, A. J., Smith, J. P., Zhang, H., Morse, E., Kaplan, R. N., & Mackall, C. L. (2014). Disruption of CXCR2-mediated MDSC tumor trafficking enhances anti-PD1 efficacy. Science translational medicine, 6(237), 237ra67.

Each publication cited in this specification is hereby incorporated by reference in its entirety for all purposes.

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention, which will be limited only by the appended claims

As used herein the singular forms “a”, “and”, and “the” include plural referents unless the context clearly dictates otherwise. Thus, for example, reference to “a cell” includes a plurality of such cells and reference to “the culture” includes reference to one or more cultures and equivalents thereof known to those skilled in the art, and so forth. All technical and scientific terms used herein have the same meaning as commonly understood to one of ordinary skill in the art to which this invention belongs unless clearly indicated otherwise.

1. A method of treating cancer, the method comprising:
contacting a population of cells comprising targeted cancer cells with a combination of (i) an agent that blockades CD47 activity; and (ii) an inhibitor of CXCR2, in a dose effective to reduce growth of the cancer.

2. The method of claim 1, wherein the contacting is performed on an individual mammal in vivo.

3. The method of claim 1, wherein the treatment provides for increased overall survival of the individual.

4. The method of claim 1, wherein reduction of tumor growth is enhanced relative to the reduction observed with a monotherapy of agent (i) or (ii) administered as a monotherapy.

5. The method of claim 1, wherein the number of granulocytic-myeloid derived suppressor cells (G-MDSC) present in the tumor microenvironment of the individual is reduced.

6. The method of claim 1, wherein the CXCR2 inhibitor is selected from Navaraxin, SB225002, SB265610, AZD5069, Danirixin, Reparixin, SX-682, Elubirixin, NSC 157449, MK-7123, and QBM076.

7. The method of claim 1, wherein the CXCR2 inhibitor is orally administered.

8. The method of claim 1, wherein the effective dose is from about 10 mg/kg up to about 100 mg/kg.

9. The method of claim 1, wherein the agent that agent that blockades CD47 activity is an anti-CD47 antibody, optionally comprising an IgG4 Fc region, optionally Magrolimab.

10-11. (canceled)

12. The method of claim 9, wherein the anti-CD47 antibody is selected from the group consisting of CC-90002, IBI188, IBI322, SRF231, AO-176, IMC-002, Lemzoparlimab, AK117, SHR-1603, ZL-1201, IBI-322, HX-009, and TG-1801.

13. The method of claim 1, wherein the agent that blockades CD47 activity is a polypeptide comprising a CD47-binding domain of SIRPα or variant thereof, optionally selected from the group consisting of TTI-621, TTI-622, ALX148, IMM01, IMM0306, IMM2902, and JMT601.

14. (canceled)

15. The method of claim 1, wherein the agent that blockades CD47 activity is an anti-SIRPα antibody, optionally CC-95251 or BI765063.

16. (canceled)

17. The method according to claim 1, wherein the mammal is a mouse.

18. The method according to claim 1, wherein the mammal is a human.

19. The method of claim 1, wherein the cancer is a solid tumor.

20. The method of claim 19, wherein the solid tumor is a melanoma.

21. The method of claim 19, wherein the solid tumor is an ovarian cancer.

22. The method of claim 1, wherein the combination of (i) an agent that blockades CD47 activity; and (ii) an inhibitor of CXCR2 provides for a synergistic effect in the reduction of cancer growth relative to the administration of (i) or (ii) as a monotherapy.

23. A method of treating an individual with an inflammatory disease associated with myeloid derived suppressor cells, the method comprising:
contacting the individual with a combination of (i) an agent that blockades CD47 activity; and (ii) an inhibitor of CXCR2, in a dose effective to reduce inflammation.
